SEQUENCE LISTING (8 sequences)

SEQ ID NO: 1 - DNA, 24 bp, Artificial Sequence
  Primer
gtctttgaggaggaaggcgatatt

SEQ ID NO: 2 - DNA, 25 bp, Artificial Sequence
  primer
agttaggtccatcaaatgggttgtt

SEQ ID NO: 3 - DNA, 24 bp, Artificial Sequence
  primer
gggctctgaatggttatgacattc

SEQ ID NO: 4 - DNA, 23 bp, Artificial Sequence
  primer
agcgctcagtctcttcacctctt

SEQ ID NO: 5 - DNA, 20 bp, Artificial Sequence
  primer
gacctgatccgtccacaaac

SEQ ID NO: 6 - DNA, 21 bp, Artificial Sequence
  primer
gtgggaaatgccgcagatatc

SEQ ID NO: 7 - DNA, 20 bp, Artificial Sequence
  primer
aagggctcatgaccacagtc

SEQ ID NO: 8 - DNA, 20 bp, Artificial Sequence
  primer
cagggatgatgttctgggca

(19) United States
(12) Patent Application Publication
(10) Pub. No.: US 2022/0111125 A1
(43) Pub. Date: Apr. 14, 2022

(54) SYSTEMS AND METHODS FOR MAKING BIOMATERIALS WITH TARGET PROPERTIES

(71) Applicant: THE ROYAL INSTITUTION FOR THE ADVANCEMENT OF LEARNING/MCGILL UNIVERSITY, Montreal (CA)

(72) Inventors: Showan N. NAZHAT, Montreal (CA); Gabriele GRIFFANTI, Montreal (CA); Ehsan REZABEIGI, Montreal (CA); William C. LEPRY, Montreal (CA)

(21) Appl. No.: 17/416,009

(22) PCT Filed: Dec. 19, 2019
(86) PCT No.: PCT/CA2019/051867

§ 371 Date: Jun. 18, 2021

Related U.S. Application Data
(60) Provisional application No. 62/782,055, filed on Dec. 19, 2018.
(60) Provisional application No. 62/877,515, filed on Jul. 23, 2019.

(51) Int. Cl. A61L 27/52; A61L 27/24; A61L 27/36; B33Y 40/10; B29C 64/314

(52) U.S. Cl. CPC A61L 27/52; A61L 27/24; A61L 27/3691; B33Y 10/00; B29C 64/314; A61L 2420/02; B33Y 40/10

(57) ABSTRACT

A method for making a biomaterial with a target property, the method comprising: obtaining a precursor biomaterial in a precursor biomaterial vessel, and a biomaterial vessel for compacting the precursor biomaterial therein, wherein a relative reduction in a given dimension of the precursor biomaterial in the precursor biomaterial vessel relative to the given dimension in the formed biomaterial in the biomaterial vessel (compaction factor) is based on the target property of the biomaterial and a change in the property of the biomaterial with the compaction factor.

### CROSS-REFERENCE

The present application claims convention priority to United States Provisional Patent Application No. 62/782,055, filed Dec. 19, 2018, entitled “SYSTEMS AND METHODS FOR MAKING BIOMATERIALS WITH TARGET PROPERTIES”, and U.S. Provisional Patent Application No. 62/877,515, filed Jul. 23, 2019, entitled “SYSTEMS AND METHODS FOR MAKING BIOMATERIALS WITH TARGET PROPERTIES” which are incorporated by reference herein in their entirety.

### FIELD

The present technology relates to systems and methods for making biomaterials with target properties.

### BACKGROUND

Biomaterials have many uses including the augmentation or replacement of soft and hard tissues in humans and animals, in vitro tissue models for research, testing and personalized medicine, and cell/drug/delivery devices. However, to date, the making of certain biomaterials, such as hydrogels, with target properties has not been practical due to a reliance on cell remodelling to achieve certain target properties, an inability to predict the properties of the biomaterials, or scale-up limitations.

It is an object of the present technology to ameliorate at least some of the inconveniences present in the prior art.

### SUMMARY

Embodiments of the present technology have been developed based on developers' appreciation of certain shortcomings associated with existing systems and methods for making biomaterials with target properties.

For certain applications, it would be advantageous to be able to automate or semi-automate the making of a biomaterial with a given target property. Bioprinting is generally defined as the use of 3D printing technology to produce tissue for reconstructive surgery or other medical uses.

However, current 3D bioprinting approaches are not compatible with the multi-scale 3D bioprinting of highly hydrated biomaterials with a solid phase. For example, type I collagen, the most prominent protein in connective tissues and the major structural component in numerous tissues has had limited success in the past in terms of 3D printing. Collagen-based hydrogels are particularly restricted by their narrow printability range, where protein structure, seeded cell viability, and bioactivity of incorporated biomolecules all need to be maintained within physiological boundaries. While collagen hydrogel printing has been claimed, in reality its use is severely limited because of its highly-hydrated nature, lack of structural control, low mechanical properties and premature gelation during the printing process.

Certain existing 3D bioprinting techniques rely on chemical crosslinking of collagen and other hydrogel biomaterials, which can be time consuming and impacts seeded cell viability. In particular, current 3D bioprinting technologies are limited in their ability to print varying length scales that replicate the complex hierarchical architecture of tissues. The extrusion of hydrogel precursor molecules, which rely on the post ejection assembly of biopolymers, lack control of fabrication resolution.

Inkjet-based printing on the other hand, inherently relies on low viscosity gels and low cell seeding densities, which lack the functionality of 3D tissue structures. Furthermore, laser-based bioprinting technologies are deficient in their ability to print large volumetric tissue constructs. These drawbacks become particularly apparent in the bioprinting of the fibrous, collagen-based hydrogels.

Furthermore, due to multi-factorial parameters that can affect a target property of a biomaterial, making biomaterials with predictable and controllable target properties has been a challenge.

According to certain aspects and embodiments of the present technology, these disadvantages are ameliorated. In certain embodiments, biomaterials with target properties can be made predictably, efficiently, and in a manner that allows tailoring of the target properties and scale-up. The methods of making the biomaterials are amenable to automation or semi-automation, in addition to manual production, such as through 3D printing (additive manufacturing) in which the manufacturing parameters can be set to produce required target properties of the biomaterial. Furthermore, composite three-dimensional structures can be produced using such biomaterial building blocks, in which the biomaterial building blocks are the same as one another or different to one another.

Broadly, developers have determined that in making a biomaterial from a precursor biomaterial using a compaction method, an extent of the compaction can be used to tailor a target property of the biomaterial. Such compaction methods have been described previously in PCT/CA2013/050615 filed Feb. 9, 2015, the contents of which are incorporated herein by reference. The compaction of a precursor biomaterial to produce the biomaterial, involves a change, generally a reduction, in a physical dimension such as a volume, a surface area, a height, a width, or a depth of the biomaterial. The compaction may comprise a confinement or a compaction of the precursor biomaterial whilst allowing fluid expulsion. Surprisingly, developers have noted that certain target properties of the biomaterial can be controlled and/or predicted by controlling the extent of the reduction in the physical dimension during compaction.

In certain embodiments, the present methods and systems are suitable to be applied to biomaterials having a solid phase and a liquid phase. In certain embodiments, the solid phase comprises fibrils (elongate solid structures). In this case, the fibrillar alignment, orientation and content can be controlled in certain embodiments. In certain embodiments, the present methods and systems are suitable for maintaining cell viability. According to certain embodiments, cellular alignment, elongation and orientation can also be controlled.

From a broad aspect, there is provided a method for making a biomaterial with a target property, the biomaterial comprising a hydrogel having a solid phase and a liquid phase, the method comprising: determining a compaction factor to be applied to the precursor biomaterial for forming the biomaterial based on a target property of the biomaterial, the compaction factor comprising a reduction in a given dimension of the precursor biomaterial relative to the given dimension in the formed biomaterial, the determining the compaction factor being based on a change in the property of the biomaterial with a change in the given dimension; and determining one or more of a value of the given dimension of the precursor biomaterial and a value of the given dimension of the formed biomaterial based on the determined compaction factor.

In certain embodiments, the target property is predetermined. In certain embodiments, the relationship between the change in the property of the biomaterial with the change in the given dimension is predetermined. In these cases, in order to obtain a biomaterial with a target property, the compaction factor can be applied to determine the extent of compaction required to obtain the target property. Applying the target property may comprise either applying the compaction factor to the dimension of the precursor biomaterial or to the dimension of the biomaterial.

The method may be executable by a processor of a computer system operatively connectable to a bio-printing system for forming the biomaterial from a precursor biomaterial through a compaction process. In other embodiments, the method may be at least partially automated, or not automated.

In certain embodiments, the method further comprises sending instructions to the bio-printing system for forming the biomaterial based on the determined one or more of:
the determined value of the given dimension of the precursor biomaterial, and
the determined value of the given dimension of the formed biomaterial. The instructions may cause the bio-printing system to aspirate at least a portion of the solid phase of the precursor biomaterial from a precursor biomaterial vessel into a biomaterial vessel to form the biomaterial with the reduction in the given dimension, the biomaterial vessel having a smaller value of the given dimension than a value of the given dimension of the precursor biomaterial vessel.

In certain embodiments, the method further comprises sending instructions to the bio-printing system to eject the formed biomaterial from the biomaterial vessel. The method may comprise causing one or more of an x-direction, a y-direction or a z-direction of the biomaterial vessel during its ejection. This can help in the creation of three-dimensional aggregate structures using one or more compacted biomaterials with the target properties.

In certain embodiments, the method further comprises causing selection of a given precursor biomaterial vessel from a kit of precursor vessels, the given precursor biomaterial vessel having the determined value of the given dimension of the precursor biomaterial. In certain embodiments, the method further comprises causing selection of a given biomaterial vessel from a kit of biomaterial vessels, the given biomaterial vessel having the determined value of the given dimension of the formed biomaterial.

In certain embodiments, the method further comprises receiving input of the target property of the biomaterial. The method may further comprise receiving input of a target value of the given dimension of the precursor biomaterial, and determining a value of the given dimension of the biomaterial based on the determined compaction factor.

In certain embodiments, the target property is one or more of: an extent of alignment of a solid phase in the biomaterial, a content of the aligned phase in the biomaterial, a content of the solid phase in the biomaterial, a distribution of the aligned phase in the biomaterial, a mechanical property of the biomaterial, and a cell-independent contraction property of the biomaterial.

In certain embodiments, the biomaterial has cells incorporated therein, and the target property is one or more of: an orientation of the cells incorporated in the biomaterial, an alignment of the cells incorporated in the biomaterial, a distribution of the cells in the biomaterial, cell activity in the biomaterial, and a cell-induced contraction property of the biomaterial. The method may further comprise causing the seeding of cells into the precursor biomaterial, such as into a starting solution for the precursor. Cell activity may include metabolic activity, contractile activity, and the like.

In certain embodiments, the given dimension is one or more of: a cross-sectional surface area of the precursor biomaterial and the biomaterial; a diameter of the precursor biomaterial and the biomaterial; a volume of the precursor biomaterial and the biomaterial; a surface area of a precursor biomaterial vessel in contact with the precursor biomaterial; and a surface area of a biomaterial vessel in contact with the biomaterial.

In certain embodiments, the method further comprises causing the display on a screen associated with the computer system of one or more of: the determined compaction factor, the determined value of the given dimension of the precursor biomaterial, and the determined value of the given dimension of the formed biomaterial based.

In certain embodiments, the compaction factor is less than about 98.6% reduction in a cross-sectional surface area of the precursor biomaterial compared to the cross-sectional surface area of the formed biomaterial, and optionally between about 88% and 98.6% reduction in the cross-sectional surface area of the precursor biomaterial compared to the cross-sectional surface area of the formed biomaterial.

In certain embodiments, the target property is a solid phase content of the biomaterial, the determining the compaction factor is based on an increase in the solid phase content of the biomaterial with an increase in the compaction factor. The target property may be a solid phase alignment of the biomaterial, and the determining the compaction factor based on an increase in the solid phase alignment of the biomaterial with an increase in the compaction factor. The target property may be a tensile property of the biomaterial, and the determining the compaction factor based on an increase in the tensile property of the biomaterial with an increase in the compaction factor. The target property may be a strength property of the biomaterial, and the determining the compaction factor based on an increase in the strength property of the biomaterial with an increase in the compaction factor. The target property may be a toughness property of the biomaterial, and the determining the compaction factor based on an increase in the toughness property of the biomaterial with an increase in the compaction factor. The target property may be a cell-induced matrix contraction property of the biomaterial, and the determining the compaction factor based on an increase in the contraction property of the biomaterial with an increase in the compaction factor. The target property may be an alignment of cells incorporated in the biomaterial, and the determining the compaction factor based on an increase in the cell alignment in the biomaterial with an increase in the compaction factor. The target property may be an elongation of cells incorporated in the biomaterial, and the determining the compaction factor based on an increase in the cell elongation in the biomaterial with an increase in the compaction factor. The target property may be an increase of metabolic activity of cells incorporated in the biomaterial, and the determining the compaction factor based on an increase in the metabolic activity with a decrease in the compaction factor. The target property may be an increase of contractile behaviour of cells incorporated in the biomaterial, and the determining the compaction factor based on an increase in the contractile behaviour of cells with a decrease in the compaction factor.

In certain embodiments, the method of forming the biomaterial comprises reducing the given dimension of the precursor biomaterial whilst allowing fluid expulsion from the precursor biomaterial to form the biomaterial.

In certain embodiments, the given dimension is a cross-sectional area of the precursor biomaterial in a precursor biomaterial vessel, and reducing the given dimension comprises causing the precursor biomaterial to flow from the precursor biomaterial vessel into a biomaterial vessel, the biomaterial vessel having a smaller cross-sectional diameter than the precursor biomaterial vessel. The given dimension may be a cross-sectional area, and the compaction factor may comprise (a cross-sectional area value of the precursor biomaterial minus a cross-sectional area value of the formed biomaterial)/the cross-sectional area value of the precursor biomaterial×100.

In certain embodiments, the biomaterial comprises one or more hydrogels selected from: collagen, hyaluronan, chitosan, fibrin, gelatin, silk fibroin, alginate, agarose, chondroitin sulphate, polyacrylamide, polyethylene glycol (PEG), poly vinyl alcohol (PVA), polyacrylic acid (PAA), hydroxy ethyl methacrylate (HEMA), polyanhydrides, poly(propylene fumarate) (PPF). In certain embodiments, the biomaterial comprises a hydrogel-borate hybrid.

In certain embodiments, the borate is a two, three or four component borate, the components selected from borate, calcium oxide, sodium hydroxide, and calcium oxide. In certain embodiments, the borate comprises a four component borate comprising: 6.1% B₂O₃-26.9% CaO-24.4% Na₂O-2.6% P₂O₅ in mol %.

From another aspect, there is provided a system for making a biomaterial comprising a hydrogel having a solid phase and liquid phase with a target property, the system comprising: a bio-printing system for forming the biomaterial from a precursor biomaterial through a compaction process; a computer system having a processor and operatively connectable to the bio-printing system, the processor arranged to execute a method comprising: determining a compaction factor to be applied to the precursor biomaterial for forming the biomaterial based on a target property of the biomaterial, the compaction factor comprising a reduction in a given dimension of the precursor biomaterial relative to the given dimension in the formed biomaterial, the determining the compaction factor being based on a change in the property of the biomaterial with a change in the given dimension; and determining one or more of a value of the given dimension of the precursor biomaterial and a value of the given dimension of the formed biomaterial based on the determined compaction factor.

In certain embodiments, the bio-printing system comprises: a pump module for applying a pressure to a precursor biomaterial to compact the precursor biomaterial into a biomaterial vessel, and optionally a sage module for enabling relative movement between the precursor biomaterial and the biomaterial.

In certain embodiments, the system further comprises a precursor biomaterial vessel for holding a precursor biomaterial, and a biomaterial vessel for compacting the precursor biomaterial therein.

In certain embodiments, the system further comprises a kit of one or more precursor biomaterial vessels and biomaterial vessels, at least some of the precursor biomaterial vessels and biomaterial vessels of the kit having different given dimensions to one another. The precursor biomaterial vessels may be pre-loaded with precursor biomaterial or with a starting solution for making a precursor biomaterial.

From another aspect, there is provided a method for making a biomaterial with a target property, the method comprising: obtaining a precursor biomaterial in a precursor biomaterial vessel, and obtaining a biomaterial vessel for compacting the precursor biomaterial therein, wherein a relative reduction in a given dimension of the precursor biomaterial in the precursor biomaterial vessel relative to the given dimension in the formed biomaterial in the biomaterial vessel (compaction factor) is based on the target property of the biomaterial and a change in the property of the biomaterial with the compaction factor. The method may further comprise compacting the precursor biomaterial into the biomaterial vessel to form a biomaterial by one or more of expulsion of fluid and application of pressure.

In certain embodiments, the method further comprises ejecting the biomaterial from the biomaterial vessel, and optionally applying pressure to eject the biomaterial from the biomaterial vessel.

In certain embodiments, the method further comprises moving the biomaterial vessel in one or more of an x-direction, a y-direction or a z-direction during the ejection of the biomaterial.

In certain embodiments, the method further comprises selecting one or more of the precursor biomaterial vessel and the biomaterial vessel from a kit.

In certain embodiments, the target property is one or more of: an extent of alignment of a solid phase in the biomaterial, a content of the aligned phase in the biomaterial, a content of the solid phase in the biomaterial, a distribution of the aligned phase in the biomaterial, a mechanical property of the biomaterial, and a cell-independent contraction property of the biomaterial.

In certain embodiments, the biomaterial has cells incorporated therein, and the target property is one or more of: an orientation of the cells incorporated in the biomaterial, a distribution of the cells in the biomaterial, cell activity in the biomaterial, and a cell-induced contraction property of the biomaterial.

In certain embodiments, the given dimension is one or more of: a cross-sectional surface area of the precursor biomaterial and the biomaterial; a diameter of the precursor biomaterial and the biomaterial; a volume of the precursor biomaterial and the biomaterial; a surface area of a precursor biomaterial vessel in contact with the precursor biomaterial; and a surface area of a biomaterial vessel in contact with the biomaterial.

In certain embodiments, the compaction factor is less than about 98.6% reduction in a cross-sectional surface area of the precursor biomaterial compared to the cross-sectional surface area of the formed biomaterial, and optionally between about 88% and 98.6% reduction in the cross-sectional surface area of the precursor biomaterial compared to the cross-sectional surface area of the formed biomaterial.

In certain embodiments, the given dimension is a cross-sectional area of the precursor biomaterial in the precursor biomaterial vessel, and reducing the given dimension comprises causing the precursor biomaterial to flow from the precursor biomaterial vessel into the biomaterial vessel, the biomaterial vessel having a smaller cross-sectional diameter than the precursor biomaterial vessel. The given dimension may be a cross-sectional area, and the compaction factor comprises (a cross-sectional area value of the precursor biomaterial minus a cross-sectional area value of the formed biomaterial)/the cross-sectional area value of the precursor biomaterial×100.

In certain embodiments, the biomaterial comprises one or more hydrogels selected from: collagen, hyaluronan, chitosan, fibrin, gelatin, silk fibroin, alginate, agarose, chondroitin sulphate, polyacrylamide, polyethylene glycol (PEG), poly vinyl alcohol (PVA), polyacrylic acid (PAA), hydroxy ethyl methacrylate (HEMA), polyanhydrides, poly(propylene fumarate) (PPF).

In certain embodiments, the biomaterial comprises a hydrogel-borate hybrid. In certain embodiments, the borate is a two, three or four component borate, the components selected from borate, calcium oxide, sodium hydroxide, and calcium oxide. In certain embodiments, the borate comprises a four component borate comprising: 6.1% B₂O₃-26.9% CaO-24.4% Na₂O-2.6% P₂O₅ in mol %.

In certain embodiments, the method further comprises one or more of: modulating the pH of the precursor biomaterial before compaction; adding bioactive particles, optionally borate glass particles, to the precursor biomaterial before compaction; and modulating the temperature of the precursor biomaterial before compaction. The borate glass particles may be added without requiring the addition of sodium hydroxide (NaOH).

In certain embodiments, the method further comprises modifying a surface roughness of an interior wall of the precursor biomaterial vessel and/or the biomaterial vessel.

From another aspect, there is provided a compacted biomaterial obtained using certain embodiments of the method as described and claimed herein.

From a yet further aspect, there is provided a kit for making a biomaterial with a target property, the kit comprising: precursor biomaterial vessels, and biomaterial vessels for compacting a precursor biomaterial therein, at least some of the precursor biomaterial vessels and biomaterial vessels of the kit having different given dimensions to one another.

In certain embodiments, the precursor biomaterial vessels are pre-loaded with precursor biomaterial or with a starting solution from which the precursor biomaterial is derived.

In certain embodiments, the biomaterial vessels each have relative surface areas which are between about 88% and 98.6% less than the cross-sectional surface areas of the precursor biomaterial vessels. The biomaterial vessels may be capillaries having an open lower end and an open upper end. In certain embodiments, the plurality of biomaterial vessels can be received one inside another to create an annular lumen into which the precursor biomaterial can be received.

From another aspect, there is provided a biomaterial comprising a hydrogel having a solid phase and a liquid phase, the biomaterial having a tubular configuration of single piece construction, the biomaterial having been obtained by compacting at least a portion of a solid phase of a precursor biomaterial into a biomaterial vessel having an annular lumen, the liquid phase content of the biomaterial being less than a liquid phase content of the precursor biomaterial. The biomaterial may be of continuous construction.

In certain embodiments, the biomaterial does not include a cross-linked component. In certain embodiments, the tubular configuration has one or more of the following dimensions: an external diameter of about 100 microns to about 2 mm, a wall thickness of about 50 microns to about 500 microns, and a length of about 5 mm to about 30 mm.

In certain embodiments, the precursor biomaterial is a collagen gel derived from an isolated collagen solution. In certain embodiments, the biomaterial further comprises boron or boron ions, optionally wherein the boron or boron ions derive from a borate glass included in the precursor biomaterial. In certain embodiments, the biomaterial further comprises one or more of calcium, sodium or phosphate ions, optionally wherein the calcium, sodium or phosphate ions derive from a borate glass included in the precursor biomaterial.

From another aspect, there is provided use of a biomaterial for one or more of: replacing or augmenting soft or hard tissue in humans or animals; as an implanted device; as a three dimensional in vitro construct; and as a drug delivery vehicle.

From a yet further aspect, there is provided a method of making a mineralizable biomaterial, the method comprising adding a bioactive glass to a precursor biomaterial solution in an amount sufficient to modulate a pH of the precursor biomaterial solution, and allowing the precursor biomaterial solution to gel. The bioactive glass may be a soluble glass which releases ions that can modulate a pH of the precursor biomaterial solution. The bioactive glass may be a borate glass, and optionally wherein the borate glass may include one or more of a calcium oxide component, a sodium oxide component and a phosphate component. The bioactive glass may be a sol-gel derived borate glass.

Possible uses for the biomaterials made using certain embodiments of the present technology include in vitro cell culturing, personalized medicine (providing a 3D biomaterial scaffold for testing of drug efficacy using a patient's own cells), implantable or injectable biomaterials for cell/drug/other active agent delivery or as a filler material.

For example, three-dimensional cell culture is a critical tool in the pharmaceutical industry enabling high throughput testing in drug discovery and safety screening. More widely, it is also impacting our understanding of cancer diagnosis and treatment mechanisms, providing an animal-free platform in the safety and toxicology testing of chemicals and cosmetics, as well as advancing stem cell research towards clinical applications in regenerative medicine. Three-dimensional cell cultures aim to mimic the physical structure of extracellular matrix of tissues, thereby facilitating in vivo-like cell-matrix communications and cell-cell interactions. Compared to 2D monolayer cultures, 3D matrices provide more physiologically relevant assays in understanding critical cellular functions such as viability, morphology, proliferation, differentiation and migration. To this end, the composition, 3D assembly, and resulting mesoscale structure of the 3D in vitro tissue model are critical to successfully mimic the native tissue itself.

According to certain embodiments, 3D biomaterials can be made which mimic the multiscale spatial resolution of native extracellular matrices. These 3D biomaterials can be 3D printed using an automated method and system.

Embodiments of the present technology can be used to make biomaterials with target properties of various geometries and sizes. For example, tubular hydrogel biomaterials having a solid phase weight percentage comparable to that of human tissue can be made. These tubular hydrogel constructs are of a single construction (i.e. they have no join seam). Possible uses for these biomaterials include bile ducts, urethra, cardiovascular vessels, etc. Furthermore, by means of certain embodiments, the solid phase and cellular alignment and orientation in the tubular biomaterials can be controlled. For example, the solid and/or cellular alignment can be made to differ across a thickness or length of the tubular biomaterial.

In the context of the present specification, unless expressly provided otherwise, a computer system may refer, but is not limited to, an “electronic device”, an “operation system”, a “system”, a “computer-based system”, a “controller unit”, a “control device” and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives.

In the context of the present specification, a “database” is any structured collection of data, irrespective of its particular structure, the database management software, or the computer hardware on which the data is stored, implemented or otherwise rendered available for use. A database may reside on the same hardware as the process that stores or makes use of the information stored in the database or it may reside on separate hardware, such as a dedicated server or plurality of servers.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 is a schematic illustration of a system for making a biomaterial comprising a computer system and a bio-printing system, according to certain embodiments of the present technology;

FIG. 2 is a schematic illustration of the computer system of FIG. 1, according to certain embodiments of the present technology;

FIG. 3 is a schematic illustration of the bio-printing system of FIG. 1 in: an initial step (FIG. 3A), a compaction step (FIG. 3B), and an ejection step (FIG. 3C), according to certain embodiments of the present technology;

FIG. 4A-4C are schematic illustrations of the bio-printing system of FIG. 1 during one or more ejection steps, according to certain embodiments of the present technology;

FIG. 5 is a schematic illustration of a precursor biomaterial vessel and a biomaterial vessel of the bio-printing system of FIG. 1, when viewed in cross-section (FIG. 5A), and top plan view (FIG. 5B), according to certain embodiments of the present technology;

FIG. 6 is a schematic illustration of another embodiment of the biomaterial vessel of FIG. 5, when viewed in cross-section (FIG. 6A) and top plan view (FIG. 6B), according to certain embodiments of the present technology;

FIG. 7 is a schematic illustration of the biomaterial vessel of FIG. 6 when viewed from the side, in use, according to certain embodiments of the present technology;

FIG. 8 is a schematic illustration of a plurality of precursor biomaterial vessels of the bio-printing system of FIG. 1, when viewed from the top, according to certain embodiments of the present technology;

FIG. 9 is a schematic illustration of a method according to certain embodiments of the present technology;

FIG. 10 is a schematic illustration of a method according to certain other embodiments of the present technology;

FIG. 11 shows (A) biomaterial vessels used in Example 1, (B) biomaterials obtained using embodiments of the present technology in Example 1, (C) scanning electron micrographs of the biomaterials, and (D) higher magnification scanning electron micrographs of the surfaces of the biomaterials, according to certain embodiments of the present technology;

FIG. 12 illustrates the solid phase weight percent of the biomaterials made with varying compaction factors of Example 1, according to certain embodiments of the present technology;

FIG. 13 illustrates solid phase direction of the biomaterials made with varying compaction factors of Example 1, according to certain embodiments of the present technology;

FIG. 14 illustrates dispersion index of the solid phase of the biomaterials made with varying compaction factors of Example 1, according to certain embodiments of the present technology;

FIG. 15A-C are confocal fluorescence microscopy images of cellular orientation in cell-seeded biomaterials of Example 4, according to certain embodiments of the present technology;

FIG. 16 show multiphoton confocal fluorescence microscopy images of the cell seeded biomaterials of Example 5, according to certain embodiments of the present technology;

FIG. 17 show confocal fluorescence microscopy images of cells in cell-seeded biomaterials of Example 6, according to certain embodiments of the present technology;

FIG. 18A-C shows cell behaviour in cell seeded biomaterials with different compaction factors in terms of (A) LDH release, (B) cell number, and (C) fluorescence intensity, according to certain embodiments of the present technology (Example 7);

FIG. 19A-C shows cell behaviour in cell seeded biomaterials with different compaction factors in terms of (A) fluorescence intensity in 95.33% compaction factor biomaterial, (B) fluorescence intensity in 95.40% compaction factor biomaterial, and (C) and (D) fluorescence intensity with varying compaction factor (Example 7), according to certain embodiments of the present technology;

FIG. 20 shows cell remodelling and biomaterial mechanical properties with varying compaction factors in biomaterials, according to certain embodiments of the present technology (Example 8);

FIG. 21 shows tubular configuration biomaterials (Example 9), according to certain embodiments of the present technology;

FIG. 22 shows surface area roughness of biomaterial vessels, according to certain embodiments of the present technology;

FIG. 23 shows effect of pH with borate glass addition to the precursor biomaterial, according to certain embodiments of the present technology (Example 12);

FIG. 24 are scanning electron micrographs of biomaterials of collagen fibrillized with borate glass and showing mineralization, according to certain embodiments of the present technology (Example 12);

FIG. 25 are scanning electron micrographs of a control collagen without borate glass, according to certain embodiments of the present technology (Example 12);

FIG. 26 illustrates change in shear storage modulus over time during gelling of biomaterials of collagen fibrillized with borate glass, according to certain embodiments of the present technology (Example 12);

FIG. 27 illustrates change in turbidity over time during gelling of a biomaterial with borate glass, according to certain embodiments of the present technology (Example 12);

FIG. 28 illustrates amounts of hydroxyapatite formed in biomaterials of collagen fibrillized with borate glass, according to certain embodiments of the present technology (Example 12);

FIG. 29A illustrates compression behaviour of biomaterials of collagen with immersion in SBF (Example 12); according to certain embodiments of the present technology;

FIG. 29B illustrates compression behaviour of biomaterials of collagen fibrillized with borate glass with immersion in SBF (Example 12); according to certain embodiments of the present technology;

FIG. 30 illustrates compressive modulus of biomaterials of collagen and collagen fibrillized with borate glass with immersion time in SBF, according to certain embodiments of the present technology;

FIG. 31 illustrates fibrin fibrillar density (FFD) weight % with increasing compaction factor (SAR %) for a collagen-fibrin hybrid hydrogel (Example 13), according to certain embodiments of the present technology;

FIG. 32A illustrates metabolic activity of cells seeded in biomaterials made with different compaction factors (Example 14), according to certain embodiments of the present technology;

FIG. 32B illustrates gene expression of contractile markers in cells of biomaterials made with different compaction factors (Example 14); according to certain embodiments of the present technology;

FIG. 33 illustrates a relationship between one or more of the compaction factor, compressive modulus and incorporation of cells in biomaterials (Example 15); according to certain embodiments of the present technology;

FIG. 34 illustrates compaction factor and collagen fibrillar density for different concentrations of collagen gel used to make collagen biomaterials (Example 16); according to certain embodiments of the present technology;

It should be noted that, unless otherwise explicitly specified herein, the drawings are not to scale.

### DETAILED DESCRIPTION

Certain aspects and embodiments of the present technology are directed to systems and methods for making a hydrogel biomaterial. Broadly, certain aspects and embodiments of the present technology comprise a computer-implemented method for making a biomaterial with at least one target property by determining a compaction factor, and for controlling various properties of the biomaterial through embodiments of the method. Broadly, certain other aspects and embodiments of the present technology comprise a method for making a biomaterial with at least one target property using a compaction factor. Other aspects are to kits for making the biomaterial. Some aspects are to the biomaterial made using the present methods and systems.

Notably, certain embodiments of the present technology provide biomaterials having target properties such as extent of solid phase alignment in the biomaterial, content of aligned solid phase in the biomaterial, a range of mechanical property of the biomaterial, cell number, cell orientation, cell alignment, cell elongation (polarization), a cell-independent contraction property of the biomaterial. Furthermore, in certain embodiments, the method can make a biomaterial incorporating viable cells therein with controllable target properties such as an orientation of the cells incorporated in the biomaterial, an elongation of cells, cell activity in the biomaterial, and cell-induced contraction property of the biomaterial.

Certain aspects and embodiments of the present technology are applicable to hydrogels having a solid phase and a liquid phase, including but limited to collagen, hyaluronan, chitosan, fibrin, gelatin, alginate, agarose, chondroitin sulphate, polyacrylamide, polyethylene glycol (PEG), poly vinyl alcohol (PVA), polyacrylic acid (PAA), hydroxy ethyl methacrylate (HEMA), polyanhydrides, poly(propylene fumarate) (PPF), silk fibroin hydrogels, and the like. The description below will be described in relation to a collagen based biomaterial, but is not limited to such.

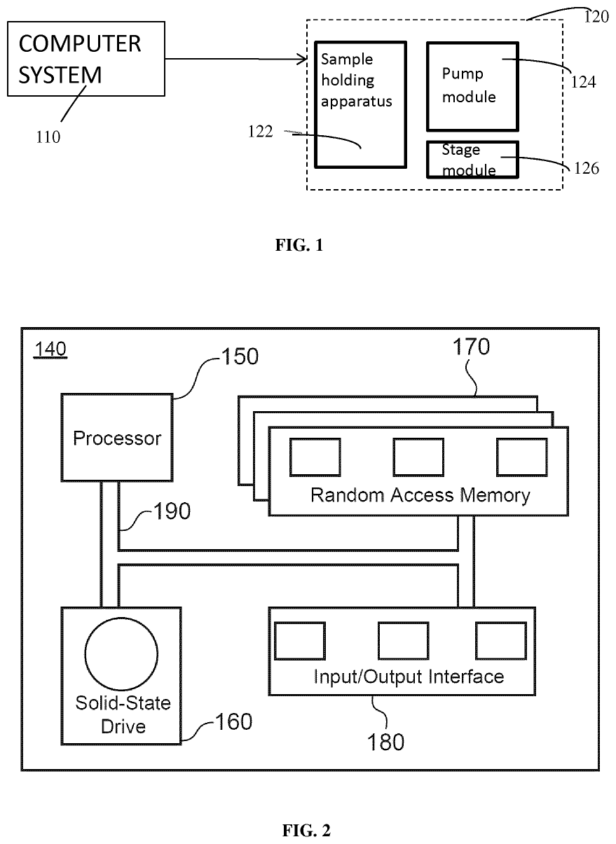

Referring initially to FIG. 1, there is illustrated one embodiment of a system 100 suitable for implementing non-limiting aspects and embodiments of the present technology. Thus, the description thereof that follows is intended to be only a description of illustrative examples of the present technology. This description is not intended to define the scope or set forth the bounds of the present technology. In some cases, what are believed to be helpful examples of modifications to the system 100 may also be set forth below. This is done merely as an aid to understanding, and, again, not to define the scope or set forth the bounds of the present technology. These modifications are not an exhaustive list, and, as a person skilled in the art would understand, other modifications are likely possible. Further, where this has not been done (i.e., where no examples of modifications have been set forth), it should not be interpreted that no modifications are possible and/or that what is described is the sole manner of implementing that element of the present technology. As a person skilled in the art would understand, this is likely not the case. In addition, it is to be understood that the system 100 may provide in certain instances simple implementations of the present technology, and that where such is the case they have been presented in this manner as an aid to understanding. As persons skilled in the art would understand, various implementations of the present technology may be of a greater complexity.

### System

Referring initially to FIG. 1, the system 100 comprises a computer system 110 operatively connected to a bio-printing system 120 for making a biomaterial 102 with target properties from a precursor biomaterial 104. The computer system 110 is arranged to implement aspects and embodiments of a method to determine various parameters for instructing the bio-printing system 120 to make the biomaterial 102.

Broadly, in the embodiment of FIG. 1, the bio-printing system 120 is arranged to form the biomaterial 102 by compaction of at least a portion of the solid phase of the biomaterial 102. In the case of collagen as the biomaterial 102, the precursor biomaterial 104 is a collagen gel which is compacted whilst allowing fluid expulsion. During the process of compaction, a given dimension of the precursor biomaterial 104 is reduced relative to the given dimension in the formed biomaterial 102. As will be explained in further detail below, the given dimension can be one or more of a respective diameter, width, cross-sectional area, volume etc. In certain embodiments described herein, the precursor biomaterial 104 compaction is achieved through a pressure-induced aspiration and ejection process. Accordingly, the bio-printing system 120 broadly comprises sample holding apparatus 122, a pump module 124 for applying pressure for the compaction and ejection processes, and a stage module 126 for enabling relative movement of different parts of the system.

### Computer System

Certain embodiments of the computer system 110 have a computing environment 140 as illustrated schematically in FIG. 2. The computing environment 140 comprises various hardware components including one or more single or multi-core processors collectively represented by a processor 150, a solid-state drive 160, a random access memory 170 and an input/output interface 180. Communication between the various components of the computing environment 140 may be enabled by one or more internal and/or external buses 190 (e.g. a PCI bus, universal serial bus, IEEE 1394 “Firewire” bus, SCSI bus, Serial-ATA bus, ARINC bus, etc.), to which the various hardware components are electronically coupled.

The random access memory 170 is configured in any known manner and arranged to store one or more of: target biomaterial 102 properties and various parameters affecting those target biomaterial 102 properties such as compaction factor, fluid loss, surface area, precursor biomaterial 104 properties, etc.

The input/output interface 180 allows enabling networking capabilities such as wire or wireless access. As an example, the input/output interface 180 comprises a networking interface such as, but not limited to, a network port, a network socket, a network interface controller and the like. Multiple examples of how the networking interface may be implemented will become apparent to the person skilled in the art of the present technology. For example, but without being limiting, the networking interface 180 may implement specific physical layer and data link layer standard such as Ethernet™, Fibre Channel, Wi-Fi™ or Token Ring. The specific physical layer and the data link layer may provide a base for a full network protocol stack, allowing communication among small groups of computers on the same local area network (LAN) and large-scale network communications through routable protocols, such as Internet Protocol (IP).

According to implementations of the present technology, the solid-state drive 160 stores program instructions suitable for being loaded into the random access memory 170 and executed by the processor 150 for executing methods according to certain aspects and embodiments of the present technology. For example, the program instructions may be part of a library or an application.

In this embodiment, the computing environment 140 and/or the computer system 110 is implemented, at least partially, in the bio-printing system 120.

In other embodiments, the computing environment 140 is implemented in a generic computer system which is a conventional computer (i.e. an “off the shelf” generic computer system). The generic computer system is a desktop computer/personal computer, but may also be any other type of electronic device such as, but not limited to, a laptop, a mobile device, a smart phone, a tablet device, or a server.

In yet other embodiments, the computing environment 140 is implemented in a device specifically dedicated to the implementation of the present technology. For example, the computing environment 140 is implemented in an electronic device such as, but not limited to, a desktop computer/personal computer, a laptop, a mobile device, a smart phone, a tablet device, a server, specifically designed for determining the orthodontic treatment. The electronic device may also be dedicated to operating other devices.

In some alternative embodiments, the computer system 110 may be hosted, at least partially, on a server. In some alternative embodiments, the computer system 110 may be partially or totally virtualized through a cloud architecture.

In the embodiments where the computing environment 140 is not implemented in the bio-printing system, the computer system 110 is operatively connected thereto.

The computer system 110 has at least one interface device (not shown) for providing an input or an output to a user of the system 100, such as a screen for providing a visual output to the user of the system, a monitor, a speaker, a printer or any other device for providing an output in any form such as image-form, written form, printed form, verbal form, 3D model form, or the like. The interface device may also comprise a keyboard and a mouse (not shown) for receiving input from the user of the system. Other interface devices for providing an input to the computer system 110 can include, without limitation, a USB port, a microphone, a camera, sensors, or the like.

The computer system 110 may be connected to other users through a server (not depicted). In some embodiments, the computing environment 140 is distributed amongst multiple systems, such as the bio-printing system and/or the server. In some embodiments, the computing environment 140 may be at least partially implemented in another system, as a sub-system for example. In some embodiments, the computer system 110 and the computing environment 140 may be geographically distributed.

As persons skilled in the art of the present technology may appreciate, multiple variations as to how the computing environment 140 is implemented may be envisioned without departing from the scope of the present technology.

### Bio-Printing System

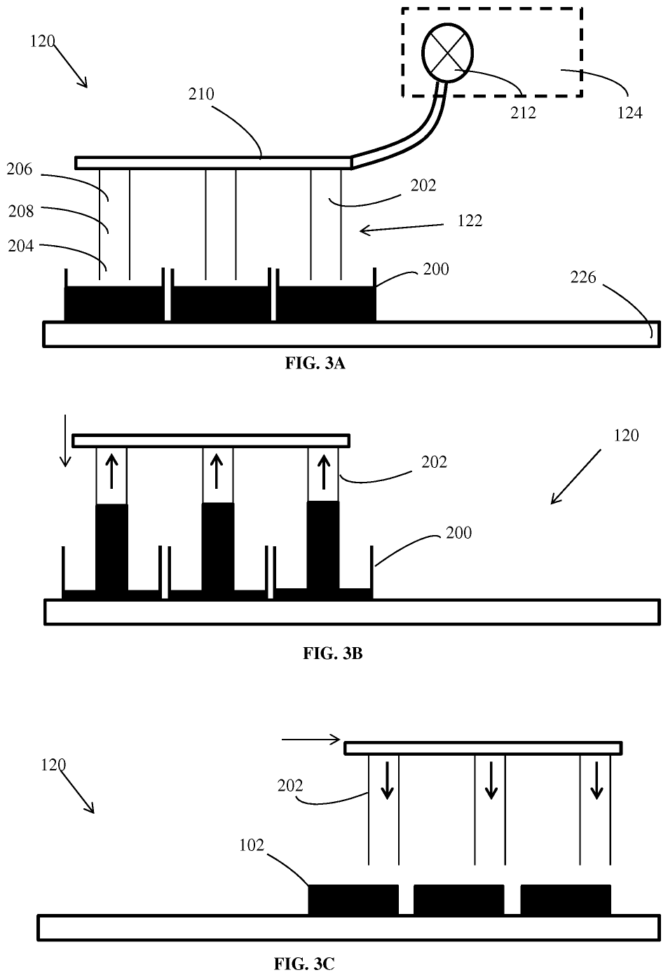

Turning now to the bio-printing system 120 which will be described in further detail with reference to FIG. 3. As mentioned above, the bio-printing system 120 is arranged to form the biomaterial 102 by compaction of the precursor biomaterial 104 through a pressure-induced aspiration/ejection process.

### Sample Holding Apparatus

As best seen in FIG. 3A, the bio-printing system 120 comprises the sample holding apparatus 122 comprising a precursor biomaterial vessel 200 for holding the precursor biomaterial 104, and a biomaterial vessel 202 for holding the formed biomaterial 102. In certain embodiments, the precursor biomaterial vessel 200 is a container having an open-face (such as a tray or a well), and the biomaterial vessel 202 is a tube such as a capillary having a lower end 204 which is open and an upper end 206 which is open, and a lumen 208 extending therethrough. In certain embodiments, the biomaterial vessel 202 is a tube with a blunt lower end 204 and/or a blunt upper end 206. A manifold 210 is provided fluidly connectable to the given upper ends 206 of the given biomaterial vessels 202 and to a pump 212 in the pump module 124 for applying negative pressure to pull at least some of the solid phase of the precursor biomaterial 104 into the lumen 208 of the biomaterial vessel 202, or to push the formed biomaterial 102 out of the biomaterial vessel 202 using positive pressure.

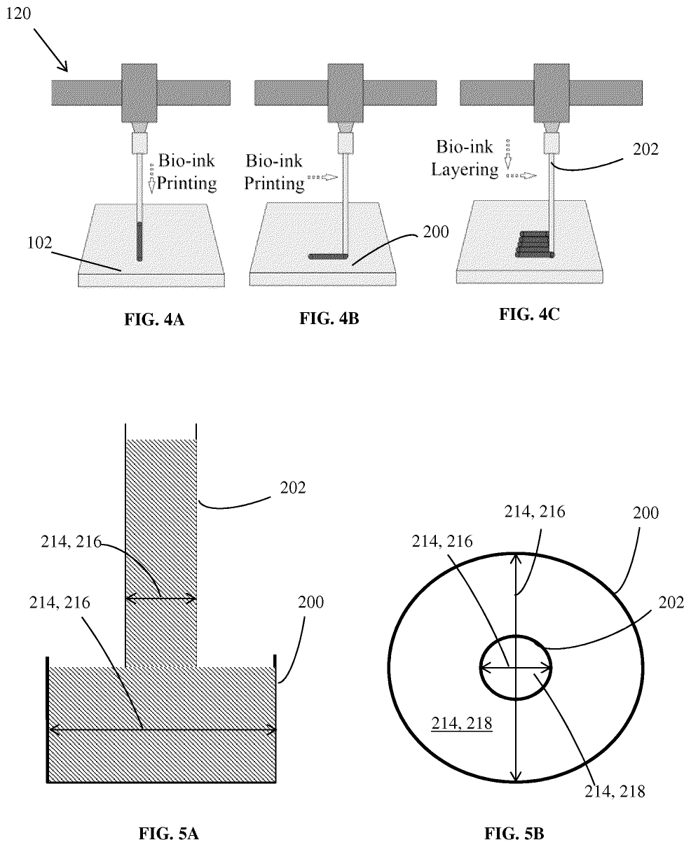

The biomaterial 102 is generally formed in the biomaterial vessel 202 by engaging the lower end 204 of the biomaterial vessel 202 with the precursor biomaterial 104 in the precursor biomaterial vessel 200 (FIG. 3A), and applying a negative pressure to pull the precursor biomaterial 104 into the biomaterial vessel 202 (FIG. 3B). In the process, some fluid is expulsed from the precursor biomaterial 104 or the formed biomaterial and is retained in the precursor biomaterial container 200. The formed biomaterial 102 in the biomaterial vessel 202 is pushed out of the biomaterial vessel 202 by application of a positive pressure through the biomaterial vessel 202 (FIG. 3C and FIG. 4). The ejection of the formed biomaterial 102 is from the lower end 204 of the biomaterial vessel 202 in these embodiments. In other embodiments, the formed biomaterial 102 is ejected from the upper end 206.

In certain embodiments, for the aspiration step, the lower end 204 of the biomaterial vessel 202 is immersed in the precursor biomaterial 104 before applying the aspirating pressure. The lower end 204 is immersed in the precursor biomaterial 104. The depth of immersion can be any appropriate depth. In certain embodiments, the depth of immersion of the lower end 204 into the precursor biomaterial 104 is from about 5% to about 30% of the depth of the precursor biomaterial 104.

As mentioned earlier, compaction is achieved in certain embodiments by providing the biomaterial vessel 202 having a smaller value of a given dimension 214 than a value of the given dimension 214 of the precursor biomaterial 104. The biomaterial vessel 202 has one or more given dimensions 214 that are smaller than those of the precursor biomaterial vessel 200 such as: diameter, width, volume and cross-sectional surface area.

FIG. 5A illustrates different given dimensions 214, comprising a diameter 216 and a cross-sectional surface area 218 on a single precursor biomaterial vessel 200 and a single biomaterial vessel 202 of FIG. 3, both having a circular-shaped cross-section. The cross-sectional surface areas 218 are on parallel respective planes. In FIG. 5B, a top plan view of the biomaterial vessel 202 positioned over the open face of the precursor biomaterial vessel 200 is shown. Both have a circular cross-sectional shape. The illustrated given dimensions 214 are the diameter 216, and the cross-sectional surface area 218. It can be seen that the cross-sectional area 218 of the biomaterial precursor vessel 200 (and hence the precursor biomaterial) is larger than the cross-sectional area 218 of the biomaterial vessel 202 (and hence the biomaterial 102). Similarly, the diameter 216 of the biomaterial precursor vessel 200 (and hence the precursor biomaterial) is larger than the diameter 216 of the biomaterial vessel 202 (and hence the biomaterial 102). In these embodiments, the diameter 216, and the cross-sectional surface area 218 are consistent through the length of the precursor biomaterial and the biomaterial.

The cross-sectional shape of the precursor biomaterial vessel 202 and the biomaterial vessel 200 need not be circular. Their cross-sectional shapes could be of any form such as quadrilateral (Example 1), triangular (not shown), oval, elliptical, pentagonal, hexagonal, and the like. Also the cross-sectional shape need not be uniform throughout a height of the vessel and can vary. For example, in certain embodiments, the biomaterial vessel 202 has a conical configuration. The cross-sectional shape of the precursor biomaterial vessel 200 and the biomaterial vessel 202 may be the same or different. In certain embodiments, one or more of the precursor biomaterial vessel 200 and the biomaterial vessel 202 may have non-symmetrical lumen shapes. For example, the lumen may include a bulbous portion. In certain embodiments, different cell populations, active agents, or even precursor hydrogels can be compacted into the biomaterial vessel 202.

In certain embodiments, the biomaterial vessel 202 may include a shaping die (not shown), such as at the lower end 204, which would further shape or create a texture on the formed biomaterial 102 as it is being expulsed from the biomaterial vessel 202. The shaping die can have any appropriate shape.

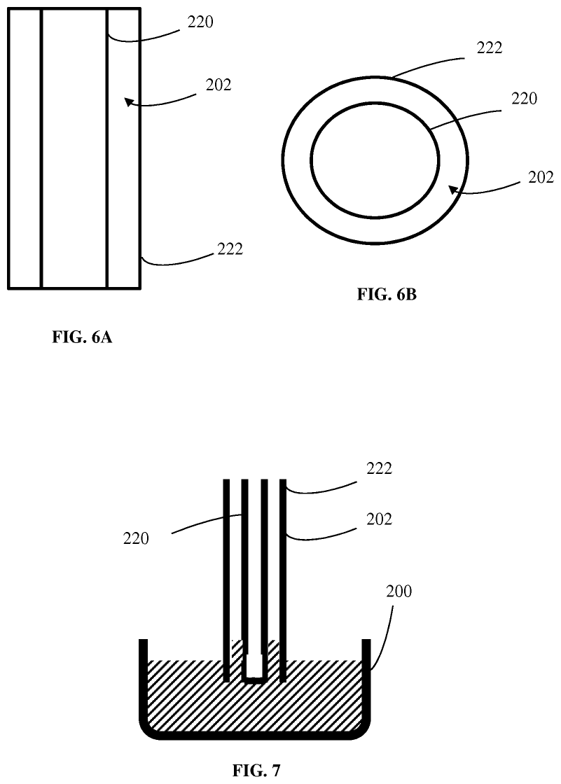

In certain embodiments, the biomaterial vessel 202 has an annular configuration (FIGS. 6A and 6B). As illustrated, the biomaterial vessel 202 in FIGS. 6A and 6B is a double-walled capillary defining an annular space between the internal walls (oppositely facing inner wall 220 and outer wall 222) for biomaterial formation therebetween The resultant formed biomaterial 102 has a tubular configuration with a wall thickness corresponding to the space between the inner wall 220 and the outer wall 222 of the biomaterial vessel 202. In certain embodiments, the biomaterial 102 produced has a continuous configuration. FIG. 7 shows the double-walled biomaterial vessel 202 in use during the aspiration step.

In other embodiments, the inner wall 220 and the outer wall 222 can be of different lengths. In certain embodiments, the inner wall 220 and the outer wall 222 may be moveable relative to each other, e.g. in an x-direction. The lumen of the biomaterial vessel 202 can be used to seed cells, drugs, active agents etc. in the biomaterial 102. This could be useful in the study of cellular migration, and to replicate tubular tissues with different cell populations across the thickness of the tubular biomaterial 102.

For example, in the case of the biomaterial vessel 202 having the annular lumen, endothelial cells may be provided to line a lumen of the tubular shaped biomaterial 102s,

In certain embodiments, one or both of the inner wall 220 and the outer wall 222 have defined surface roughnesses for attaining an alignment of the solid phase of the biomaterial 102. The inner wall 220 and the outer wall 222 may have the same or different surface roughness. A required surface roughness can be applied to the inner wall 220 and/or the outer wall 222 through any appropriate manner, such as by etching, mechanical abrasion, laser abrasion, and the like. Methods of providing an appropriate surface roughness to the inner wall and/or outer wall of the biomaterial vessel 202 are included herein.

As illustrated in FIG. 2, the sample holding apparatus 122 comprises three (3) pairs of the precursor biomaterial vessels 200 and biomaterial vessels 202. In other embodiments, more or less than the three pairs are provided. In certain embodiments, the precursor biomaterial vessels 200 are provided as an array 224 (FIG. 8) of precursor biomaterial vessels 200, such as in a well plate configuration with the wells of the well plate functioning as the precursor biomaterial vessels 200. The array 224 of precursor biomaterial vessels 200 may comprise any configuration of the precursor biomaterial vessels 200, such as 3×4, 6×6, 8×10, 8×11, 8×12, 10×10 configurations (also referred to as 6, 12, 24, 48, 96, 384 cell culture plates, etc.). Each precursor biomaterial vessel 200, in the array 224 or otherwise, may have the same or different size and/or shape to one another. Each biomaterial vessel 202 may have the same or different size and/or shape to one another.

In certain embodiments, the bio-printing system 120 is also provided with one or more kits (not shown). In certain embodiments, the kits comprise a plurality of biomaterial vessels 202 having different sizes or different configurations. In certain embodiments, the kits comprise a plurality of precursor biomaterial vessels 200 having different sizes or different configurations. In certain embodiments, the kits comprise a plurality of biomaterial vessels 202 and precursor biomaterial vessels 204. For example, in certain embodiments, the kit comprises a plurality of capillaries as the biomaterial vessels 202 having different diameters or different cross-sectional areas. In certain embodiments, the kit comprises a plurality of biomaterial vessels 202 having different cross-sectional shapes. In certain embodiments, the kit comprises a plurality of annular-walled biomaterial vessels 202 having different inner wall 220 and outer wall 222 diameters. In certain embodiments, the kits include a plurality of biomaterial vessels 202 having different internal cross-sectional surface area values. In certain embodiments, the kit comprises a plurality of precursor biomaterial vessels 200 having different values of the given dimension 214, and optionally preloaded with the precursor biomaterial 104 or with an initial component for preparing the precursor biomaterial 104 (such as the hydrogel in a pre-gelled form, such as collagen solution). Combinations of these kits are also possible. In this way, the kits, in certain embodiments, can provide the means for providing different compaction factors.

In this respect, in certain embodiments, the bio-printing system 120 is also provided with an robotic mechanism (not shown) for selecting the appropriate biomaterial vessels 202 and/or the precursor biomaterial vessels 200 from the one or more kits, and for positioning the selected biomaterial vessels 202 and/or precursor biomaterial vessels 200 in the bio-printing system 120. Selection can be achieved through any suitable identification means such as RFID tagging, imaging, barcoding and the like. Positioning can be through use of at least one robotic arm with a grabbing end, for fetching the desired vessel from the kit(s).

### Stage Module

The stage module 126 of the bio-printing system 120 enables relative movement between a given biomaterial vessel 202 and a given precursor biomaterial vessels 200 so that the formed biomaterials 102 can be deposited at a different location to the precursor biomaterial vessels 200. In certain embodiments, the relative movement is enabled through one or more of: movement of individual or grouped biomaterial vessels 202, movement of individual or grouped precursor biomaterial vessels 200 and movement of a platform 226 on which the precursor biomaterial vessels 200 are placed. Movement of any of the aforementioned components is in one or more of an x-direction, a y-direction, and a z-direction. The stage module 126 also provides a framework to allow the biomaterial vessels 202 to move in a y-direction, towards and away from the precursor biomaterial vessels 200 in order to contact the precursor biomaterial 104 with their lower end 204. In certain embodiments, the stage module 126 enables the biomaterial vessels 202 or the platform 226 to move in a z-direction. This three-dimensional relative movement also enables the bio-printing system 120 to place the formed biomaterials 102 in a manner allowing the construction of three-dimensional structures made up of individual formed biomaterials 102. For example, the formed biomaterial 102 can be placed next to each other, on top of each other, etc. In another example, the biomaterial vessel 202 can be moved spirally whilst ejection the compacted biomaterial 102 in order to form a spiraled or a pyramidal configuration of the formed biomaterial 102.

### Pump Module

The pump 212 of the pump module 124 may be any type of pump 212, such as but not limited to infusion pump (pulse or pulseless flow), pressure pump (low, mid and high pressure), vacuum pump, peristaltic pump, etc.

### Precursor Biomaterial

The precursor biomaterial 104 has a solid phase and a liquid phase and is a highly hydrated version of the biomaterial 102 to be obtained through embodiments of the present technology. In other words, the biomaterial 102 with the target properties formed through embodiments of the present technology has a lower fluid content and a higher relative solid phase content than the precursor biomaterial 104. In certain embodiments, the precursor biomaterial 104 is a collagen gel in which fibrillogenesis has already commenced. This can be achieved through fibrillogenesis of a collagen solution by allowing the collagen solution to self-assemble with or without the use of external stimuli such as heating, cooling, pH changes, cross-linkers, addition of borate glass (Example 12). By fibrillogenesis is meant a liquid to gel transition which may be spontaneous,

In certain embodiments, the precursor biomaterial 104 includes viable cells, such as any cells involved in hard and soft tissue generation, regeneration, repair and maintenance. The cells can be mammalian cells, for example, and may include mesenchymal stem cells, mesenchymal stromal cells, embryonic stem cells, bone marrow stem cell, osteoblasts, preosteoblasts, fibroblasts, muscle cells, nerve cells, Schwann cells, chondrocytes, cancer cells, immune cells, populations of cells such as from a bone marrow aspirate, and combinations of the same. The cells can be added to the precursor biomaterial 104 or to a starting solution from which the precursor biomaterial 104 is derived.

In certain embodiments, the precursor biomaterial 104 also includes one or more of drug molecules, therapeutic agents, particles, bioactive agents, osteogenic agents, osteoconductive agents, osteoinductive agents, anti-inflammatory agents, growth factors, fibroin derived polypeptide particles, and combinations of the same. Examples of particles include bioactive glass, soluble glass, resorbable calcium phosphate, hydroxyapatite, calcium carbonate, calcium sulphate, glass-ceramics, to name a few. The particles may be microspheres. They may be porous or non-porous. Therapeutic agents can include hormones, bone morphogenic proteins, antimicrobials, anti-rejection agents and the like. The drugs can be any molecules for disease, condition or symptom treatment or control, anti-inflammatory, growth factors, peptides, antibodies, vesicle for release of ions, release of gas, release of nutrients, enzymes, as well as nano carriers within the dense hydrogels. In this way, the biomaterial 102 may be used as a substance carrier or as a delivery vehicle, such as for controlled release of drugs or therapeutic agents. It is thought that sustained release may improve the success of the therapy and minimize the possible side effects. This is particularly true in the case of cancer treatment, where antineoplastic drugs are very debilitating for the patient body. Delivering the drugs, for sustained release, in the biomaterial 102, is therefore advantageous. These agents can be added to the precursor biomaterial 104 or to a starting solution from which the precursor biomaterial 104 is derived.

### Method

Turning now to the method 500 for making the biomaterial 102 with target properties. In certain embodiments, the method 500 is executed by a processor of a computer system operatively connected to a bio-printing system, such as the processor 150 of the computer system 110 and the bio-printing system 120 described and illustrated herein. In certain embodiments, the bio-printing system 120 operatively connected to the computer system 110 can differ from that described herein. In certain embodiments, the method 500 can be performed manually, at least in part.

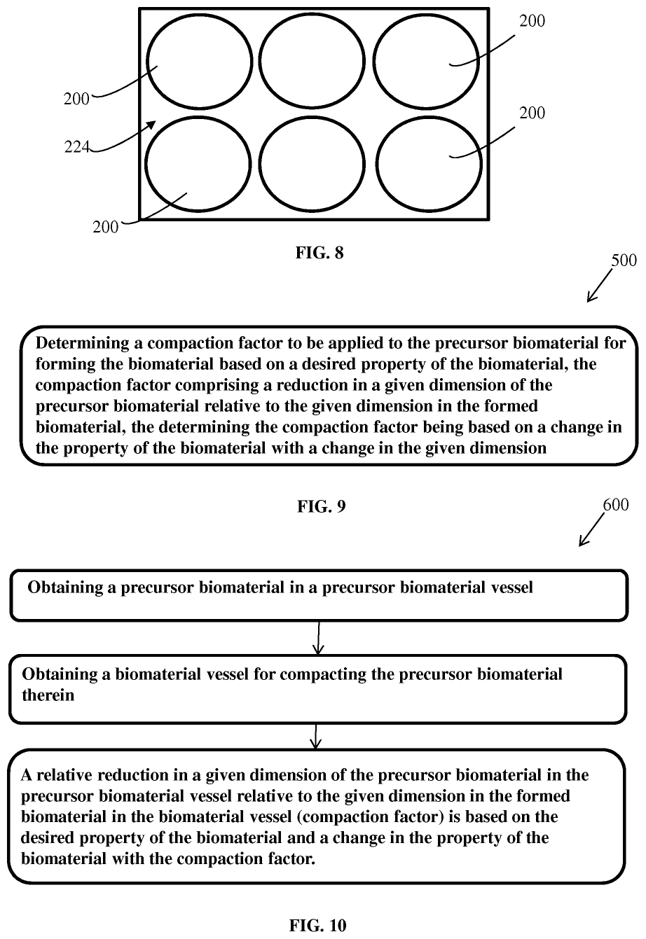

With reference now to FIG. 9, in certain embodiments the computer system 110 is configured to execute a method 500, the method 500 comprising:

Determining a compaction factor to be applied to the precursor biomaterial for forming the biomaterial based on a target property of the biomaterial, the compaction factor comprising a reduction in a given dimension of the precursor biomaterial relative to the given dimension in the formed biomaterial, the determining the compaction factor being based on a change in the property of the biomaterial with a change in the given dimension; and determining one or more of a value of the given dimension of the precursor biomaterial and a value of the given dimension of the formed biomaterial based on the determined compaction factor.

In certain embodiments, the processor 150 obtains input of the target property of the biomaterial 102, such as an extent of alignment of the solid phase in the biomaterial 102. The input can be obtained from a database or comprise a manual input by a user of the method 500.

Other target properties include, without limitation, a content of an aligned phase, a content of the solid phase in the biomaterial 102, a mechanical property of the biomaterial 102, and a cell-independent contraction property of the biomaterial 102. For biomaterials 102 with cells incorporated therein, the target property may include an orientation of the cells incorporated in the biomaterial 102, cell activity in the biomaterial 102, and a cell-induced contraction property of the biomaterial 102.

The processor 150, in certain embodiments, obtains input of a target value of the given dimension of the formed biomaterial 102. In these embodiments, the given dimension is a cross-sectional surface area of the biomaterial 102. The given dimension of the formed biomaterial 102 is related to the corresponding given dimension of the biomaterial vessel 202 i.e. the cross-sectional area of the biomaterial vessel 202, which is a capillary for example, is substantially the same as the cross-sectional area of the biomaterial 102 formed in the biomaterial vessel 202. Other possible given dimensions are a diameter of the precursor biomaterial 104 and the biomaterial 102; and a volume of the precursor biomaterial 104 and the biomaterial 102.

The processor 150 then determines a compaction factor to be applied to the precursor biomaterial 104 whilst compacting at least a portion of the solid phase of the precursor biomaterial 104 into the biomaterial vessel 202 to obtain the target property and the target value of the given dimension. The compaction factor to be applied to the precursor biomaterial 104 comprises a reduction in the value of its cross-sectional surface area during aspiration from the precursor biomaterial vessel 200 into the biomaterial vessel 202. This is related to an extent of fluid loss in certain embodiments.

Based on the determined compaction factor, the processor 150 can then determine the value of the cross-sectional surface area required for the precursor biomaterial 104 before its aspiration into the biomaterial vessel 202.

In certain embodiments, the processor 150 is provided with the target value of the given dimension of the precursor biomaterial 104. In these cases, based on the determined compaction factor, the processor 150 can determine the required value of the given dimension of the formed biomaterial 102.

The compaction factor determination is based on a change in the property of the biomaterial 102 with a change in the given dimension. Embodiments of the present technology are based on inventors' observation that an extent of the applied compaction (e.g. an extent of reduction in a surface area, diameter, volume), together with other parameters such as a surface roughness of the internal walls of the biomaterial vessel 202 and/or the precursor biomaterial vessel 200 and a pH of the precursor biomaterial 104, during the formation of the biomaterial 102 is related to certain properties of the biomaterial 102. Therefore, in certain embodiments, controlling the extent of compaction can attain certain target properties of the biomaterial 102.

Inventors have defined the extent of compaction using a “compaction factor” which can be defined as a relative reduction in a given property of the precursor biomaterial 104 when forming the biomaterial 102. This is based on a loss of at least some of the fluid contained in the precursor biomaterial 104.

The compaction factor can be expressed using the following equation:

$$Compaction factor = \frac{\begin{matrix}( Precursor Biomaterial Given \\ Dimension initial value - \\ Formed biomaterial Given \\ Dimension final value )\end{matrix}}{\begin{matrix}( Precursor Biomaterial Given \\ Dimension initial value )\end{matrix}} \times 100$$

In certain embodiments, the determination of the compaction factor is through the application of a machine learned algorithm (MLA). Accordingly, the computer system 110 or the processer 150 of FIG. 2 is arranged to implement the MLA for determining, by the MLA, the value of the given dimension of the precursor biomaterial 104 or the biomaterial 102.

The MLA may comprise, without being limitative, a non-linear regression, a linear regression, a logistic regression, a decision tree, a support vector machine, a naïve bayes, K-nearest neighbors, K-means, random forest, dimensionality reduction, neural network, gradient boosting and/or adaboost MLA.

In certain embodiments, the computer system 110 is also arranged to execute a training phase of the MLA based on various inputs, such as, but not limited to, surface area of the precursor biomaterial 102, surface area of the formed biomaterial 102, surface roughness of the biomaterial vessel 202, a pH of the precursor biomaterial 104, viscoelasticity of the precursor biomaterial 104 (e.g. modulus), and target properties of the biomaterial 102 (e.g. solid phase content, solid phase alignment, cell alignment, cell elongation, mechanical properties, etc) and the like. In some embodiments, the MLA may be trained, re-trained or further trained by the computer system 110 based on data collected, such as from the bio-printing system 120 or by other means. In other words, an output from the bio-printing system 120 or any other output can be fed back into the MLA for training or re-training.

In certain embodiments, the determination of the value of the given dimension to be applied to the precursor biomaterial 104 or the biomaterial 102 is by means of a look-up table which may be stored as a database in the RAM 170 of the computer system 110.

Various relationships between the compaction factor and the target property have been identified, some of which are listed here below and included in the Examples.

An increase in compaction factor is associated with increasing solid phase content, increasing solid phase alignment, increased cell alignment, elongated cell morphology, cell behaviour, cell remodelling, mineralization, mechanical (tensile) properties.

Additional parameters that are also associated with the target properties include a surface area roughness, pH, viscoelasticity. The inventors have observed that the inter-relationship between these different parameters is sometimes linear and sometimes non-linear.

In certain embodiments, a compaction factor of less than about 98.6% reduction in a cross-sectional surface area of the precursor biomaterial 104 (e.g. collagen gel) compared to the cross-sectional surface area of the formed biomaterial 102 is applied in the making of the biomaterial 102 by its aspiration from the precursor biomaterial 104.

In certain embodiments, a compaction factor of between about 88% and 99% reduction in the cross-sectional surface area of a collagen gel as the precursor biomaterial 104 compared to the cross-sectional surface area of the formed biomaterial 102 is utilized. In certain other embodiments, a compaction factor range of about 50% to about 99% reduction in cross-sectional area of the precursor material 104 during compaction.

In certain embodiments, the method 500 comprises the step of: sending instructions to the bio-printing system 120 for forming the biomaterial 102 based on the determined one or more of: the determined value of the given dimension of the precursor biomaterial 104, and the determined value of the given dimension of the formed biomaterial 102.

The instructions may cause the bio-printing system 120 to aspirate the precursor biomaterial 104 from the precursor biomaterial vessel 200 into a biomaterial vessel 202 to form the biomaterial 102 with the determined reduction in the given dimension, the biomaterial vessel 202 having a smaller value of the given dimension than a value of the given dimension of the precursor biomaterial vessel 200. In this respect, the processor 150 may select a suitable aspiration rate. The bio-printing system 120 is arranged to allow compaction of the solid phase of the precursor biomaterial 104 into the biomaterial vessel 202, whilst allowing at least some fluid to remain in the precursor biomaterial vessel 200 or to be expulsed from the biomaterial vessel 202.

In certain embodiments, the processor 150 causes the pump module 124 to apply pressure through the upper end of the biomaterial vessel 202 in order to compact at least a portion of the solid phase of the biomaterial precursor 104 therein. The processor 150 may also cause the stage module 126 to position the lower end of the biomaterial vessel 202 in the precursor biomaterial 104 in order to contact the precursor biomaterial 104.

Once the precursor biomaterial 104 has been compacted into the biomaterial vessel 202, the method 500 may further comprise the processor 150 causing the bio-printing system 120 to eject the precursor biomaterial 104 from the biomaterial vessel 202. In this respect, the processor 150 may cause the pump module 126 of the bio-printing system 120 to apply a suitable pressure to eject the formed biomaterial 102 from the biomaterial vessel 202.

The method 500 further comprises, in certain embodiments, sending instructions to the stage module 126 to cause a relative movement of the biomaterial vessel 202 and the platform 226 or the desired destination for the formed biomaterial 102 in order to position the formed biomaterial 102 in a desired position. In this way, three-dimensional larger structures can be made using units of the formed biomaterial 102. Adhesive, such as fibrin glue, are used in certain embodiments to attach the units of the formed biomaterial 102 to one another. Alternatively, the method 500 comprises positioning the formed biomaterials 102 for storage. Three-dimensional structures may comprise a pyramidal structure made from cylindrical biomaterial units.

In certain embodiments, before the precursor biomaterial aspiration step, the method 500 comprises sending instructions to the bio-printing system 120, based on the determined compensation factor, to select one precursor biomaterial vessel 200 or biomaterial vessel 202 having the determined value of the given dimension of the precursor biomaterial 104 or the formed biomaterial 102, respectively, from the kit of biomaterial vessels 202. In this respect, the precursor biomaterial vessels 200 in the kit may be pre-loaded with the precursor biomaterial 104. Alternatively, the method 500 comprises, in certain embodiments, causing the loading of the precursor biomaterial 104 into the selected precursor biomaterial vessel 200.

In certain embodiments, the method 500 comprises causing an initial processing of the precursor biomaterial 104, such as, one or more of:
adding cells, particles, drugs, therapeutic agents, mineralizing agents, or bioactive agents to the precursor biomaterial 104,
causing a pH change in the precursor biomaterial 104, and
causing a temperature change in the precursor biomaterial 104.

In certain embodiments, the bioactive agent is bioactive glass particles, such as borate glass particles which can both affect the pH, hence fibrillogenesis, and induce mineralization (Example 12).

In certain other embodiments, the precursor biomaterial vessel 200 and/or the biomaterial vessel 202 have an adjustable given dimension, and the method 500 comprises causing the adjustment of the given dimension to the determined value of the given dimension of the precursor biomaterial 104, or to the determined value of the given dimension of the formed biomaterial 102.

In certain embodiments, the method 500 further comprises causing the display on a screen associated with the computer system 110 or the bio-printing system 120 of one or more of: the determined compaction factor, the determined value of the given dimension of the precursor biomaterial 104, and the determined value of the given dimension of the formed biomaterial 102.

### Method for Making a Biomaterial

In certain embodiments, a method 600 for making a biomaterial with a target property comprises obtaining a precursor biomaterial 104 in a precursor biomaterial vessel 200, and obtaining a biomaterial vessel 202 for compacting the precursor biomaterial 104 therein, wherein a relative reduction in a given dimension of the precursor biomaterial 104 in the precursor biomaterial vessel 200 relative to the given dimension in the formed biomaterial 102 in the biomaterial vessel 202 (compaction factor) is based on the target property of the biomaterial 102 and a change in the property of the biomaterial 102 with the compaction factor (FIG. 10).

In certain embodiments, the method 600 is executed by the processor 150 of the computer system 140.

### EXAMPLES

The examples below are given so as to illustrate the practice of various embodiments of the present disclosure. They are not intended to limit or define the entire scope of this disclosure.

### Example 1: Generating Dense Collagen Structures

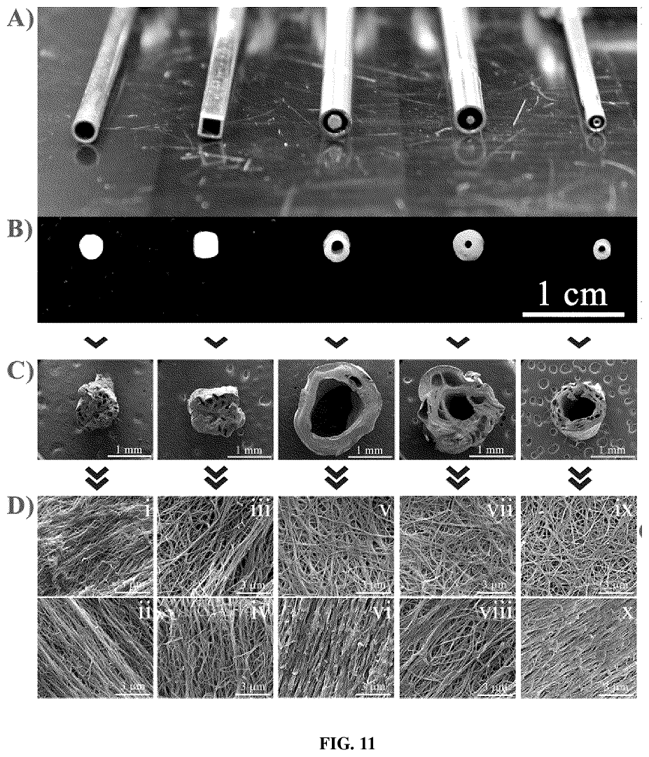

Collagen solution was used as the precursor biomaterial in a precursor biomaterial vessel, which was an open-faced tray, and aspirated into different biomaterial vessels in the form of capillaries with various configurations (FIG. 11A). The capillaries had circular or quadrilateral cross sections. Three of the circular cross-section capillaries were double-walled (annular lumen). In this example, the capillaries used were needles of certain gauge sizes.

The precursor biomaterial was a neutralized rat-tail tendon derived type I collagen solution (about 2 mg/ml) in which fibrillogenesis was initiated by incubating at 37° C. to allow for gel formation. Other concentrations of the collagen solution can be used, such as 0.5 mg/ml to about 10 mg/ml). Compacted collagen gels were formed by aspirating at least a portion of the precursor biomaterial into the various biomaterial vessels.

TABLE 1
Compaction factors applied by each of the biomaterial vessels of FIG. 11.

| | Biomaterial | Biomaterial | Biomaterial | Biomaterial | Biomaterial |
|---|---|---|---|---|---|
| | vessel 1 | vessel 2 | vessel 3 | vessel 4 | vessel 5 |
| Biomaterial | 95.03 mm² | 95.03 mm² | 95.03 mm² | 95.03 mm² | 32.17 mm² |
| precursor | | | | | |
| surface area | | | | | |
| Formed | 3.66 mm² | 3.66 mm² | 3.98 mm² | 5.15 mm² | 1.48 mm² |
| biomaterial | | | | | |
| surface area | For | For | For tubular | For tubular | For tubular |
| | cylindrical | quadrangular | dense | dense | dense |
| | dense | dense | collagen (T- | collagen (T- | collagen (T- |
| | collagen (C- | collagen (Q- | DC) | DC) | DC) |
| | DC) using | DC) using | using coaxial | using coaxial | using coaxial |
| | cylindrical | quadrangular | needle | needle | needle: |
| | needle 12G | needle 12G | 10&17G | 10&21G | 14&21G |
| Compaction | 95.33 | 95.33 | 95.81 | 94.58 | 95.40 |
| factor | | | | | |

Gross images of the resulting compacted collagen biomaterials made using embodiments of the present technology are shown in FIG. 11B. Scanning electron micrographs of the resultant collagen biomaterials are shown in FIG. 11C. Higher magnification scanning electron micrographs of the resultant collagen biomaterial surface are shown in FIG. 11D in which the compacted solid phase (fibrils) can be clearly seen.

The sizes and compaction factors (percentage reduction in precursor biomaterial surface area through compaction; also referred to as SAR in the figures) applied for each of the illustrated biomaterial vessels (left to right in FIG. 11A) are presented in Table 1. These forms were chosen to mimic the shape of cylindrically shaped volumetric monolith tissues (C-DC), continuous luminal or tubular tissues (T-DC) or the generation of highly defined quadrangular shaped viable tissue building blocks (Q-DC) to enable the layer-by-layer assembly of tissues or organs. For example, the scaling-down of these Q-DC compacted biomaterials can be useful in the context of bottom-up fabrication of complex, hierarchically structured tissues, where gels can be stacked as either acellular or cell-seeded building blocks. SEM micrographs of the cross-sections of the compacted biomaterials showed that their gross morphology was maintained after drying (FIG. 11C).

SEM images (FIG. 11D) of the surfaces of the circular and quadrilateral cross-sectional shaped-cylindrical biomaterials qualitatively showed higher extents of fibrillar alignment with an increase in compaction factor. Moreover, for the tubular compacted biomaterials, there was a striking difference in fibrillar alignment between the external and luminal surfaces, in which the fibrils were well aligned on the external surface (FIG. 11D vi, viii and x), whereas there was no preferential fibrillar alignment on their luminal surface (FIG. 11D, v, vii and ix).

### Example 2: Compaction Factor is Related to Target Properties—Solid Phase Content

The solid phase content of the formed biomaterials of Example 1 were determined by gravimetrically weighing the compacted collagen gels immediately after compaction (W[wet]), freezing the compacted collagen gels in liquid nitrogen for 3 min followed by freeze-drying overnight at 13 mT, and weighing again (W[dry]). The solid phase weight percent was calculated according to Equation 2:

$$Solid Phase Content = \frac{W [ dry ]}{W [ wet ]} \times 100 \qquad ( 2 )$$

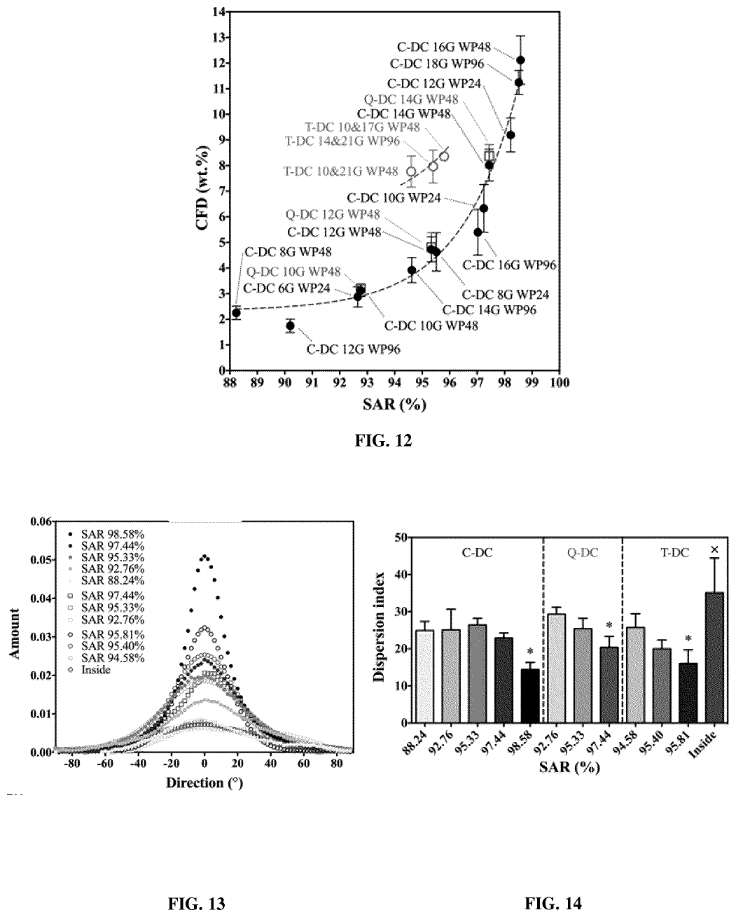

It was demonstrated that solid phase content weight % (indicated as collagen fibrillar density (CFD) along the Y-axis) of the biomaterials of Example 1 (wt. %) increased as a function of compaction factor (%) (FIG. 12).

The trend line fitted to the experimental data points is defined by Equation 3 (R²=0.92) describing the relationship between CFD and the compaction factor (for collagen gels prepared using an initial collagen concentration of 2 mg/mL with height range of 1 to 4 cm):

CFD=0.14(SAR)²−25.52(SAR)+1160 (3)

The CFD values of the annular-shaped compacted collagen gels were on average higher than those of the corresponding circular shaped compacted collagen gels, thought to be due to the more complex shear stress profiles.

It was also demonstrated that for compaction values higher than ˜98.60%, the hydrated collagen gel (biomaterial precursor) could not be completely aspirated (data not shown), suggesting that there is a limit to solid phase compaction and fluid expulsion.

The relationship between increasing compaction factor and CFD was also demonstrated for tubular collagen biomaterials made as described in Example 9 (Table 2).

TABLE 2
Relationship of CFD with varying compaction
factors of tubular collagen biomaterials.

| Compaction factor of | |
|---|---|
| tubular biomaterials (%) | CFD (wt %) |
| 95.81 | 8.35 ± 0.14 |
| 95.39 | 7.96 ± 0.64 |
| 94.58 | 7.77 ± 0.61 |
| 83.99 | 1.82 ± 0.05 |

### Example 3: Compaction Factor is Related to Target Properties—Solid Phase Alignment

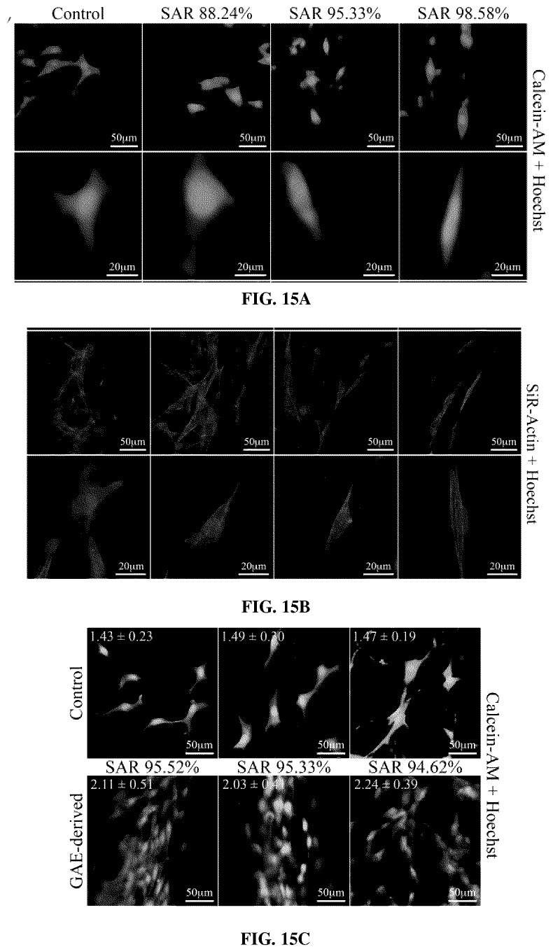

Quantification of solid phase (fibril) alignment in the compacted biomaterials of Example 1, evaluated through directionality and dispersion (FIGS. 13 and 14), which corroborated the SEM images of FIG. 11D. Solid phase alignment (fibril directionality) of the biomaterials of Example 1 was found to increase with increasing compaction factor. The fibril direction (in degrees) was measured using an image analysis software (ImageJ (NIH, USA) with the Fiji open-source plug-in) on field-emission scanning electron microscopy images (Schindelin et al, Fiji: an open-source platform for biological-image analysis, Nat Meth 9(7) (2012) 676-682, the contents of which are incorporated herein). This was confirmed by measuring mean fibril dispersion angles calculated through image analysis (FIG. 14).

Gaussian distribution of fibril directionality was narrower with increasing compaction values indicating higher fibrillar alignment (FIG. 13). Additionally, the extent of alignment appeared regulated by compaction factor when the same needle geometry was used. The corresponding dispersion indices obtained from the Gaussian distributions also supported this hypothesis (FIG. 14). On the other hand, when the same compaction factor was used, but with biomaterial vessels of different geometries, different extents of alignment were generated, suggesting biomaterial vessel geometry is also a factor.

### Example 4: Compaction Factor is Related to Target Properties—Cell Morphology

Fibroblast cells (passage 10 NIH/3T3) at 80% confluency were seeded into the collagen solution of Example 1, after collagen solution neutralization and before collagen solution gelation (i.e. before formation of the precursor biomaterial). Cells were seeded at a density of 2×10⁵ cells/mL into different volumes of the neutralized collagen solution. Embodiments of the present method were applied to the cell-seeded precursor biomaterial using different compaction factors as described in Example 1 to make compacted biomaterials.

Seeded cells were stained with the following dyes (alone or in combination) and incubated at 37° C. for 30 min prior to imaging: calcein-AM solution leading to green fluorescence for live calcium-laden cells; ethidium homodimer-1 leading to red fluorescence for compromised or dead cell nuclear content; hoechst solution (bis-benzimides) for blue fluorescence of cell nucleus; SiR-Actin for red fluorescence for actin filaments. Confocal laser scanning microscopy of the biomaterials showed that the cells remained viable throughout the biomaterial formation at days 1, 4, 7, and 10 in culture.

The effect of the present technology on the short-term responses of seeded fibroblast cells was investigated through their cellular morphology. The microarchitecture of a scaffold can be pivotal in determining aspects of cell morphology through cell-matrix interactions. Confocal laser scanning microscopy (CLSM) of Calcein-AM and Hoechst-stained cells seeded in the precursor biomaterial collagen gels revealed stellate and circular morphologies (Control in FIG. 15A). The cells seeded in compacted biomaterials were progressively more elongated with a stretched nucleus with increasing compaction factor values (compaction factor (SAR) 88.24%, 95.33% and 98.58% in FIG. 15A). Further as can be deduced from actin filament staining (FIG. 15B), the cytoskeleton of fibroblasts seeded in the precursor biomaterial collagen gels, appeared relaxed and randomly oriented in compacted biomaterials of 88.24% compaction factor. In contrast, cells were progressively more stretched and aligned along the aspiration direction when seeded in denser compacted biomaterials of 95.33 and 98.58% compaction factor.

Interestingly, cell membranes were not damaged as a consequence of the compaction mechanical forces. The rate of compaction affected the compression force that the cells had to withstand during aspiration. The flow rate was adjusted according to the compaction factor, which dictated the rate of aspiration and limited cell damage. However, the increasing time of gel aspiration due to the increased compaction factor, slowed down this compaction rate.

At day 3 in culture, the elongation ratios of the nuclei of cells seeded in collagen gels (precursor biomaterial) of different volumes were not significantly (p>0.05) different (FIG. 15C—control). After compaction, the elongation ratio of these cells increased significantly (p<0.05) compared to the control. Furthermore, similar compaction factors resulted in similar elongation ratios of seeded cells in the compacted biomaterials (FIG. 15C).

### Example 5: Compaction Factor is Related to Target Properties—Cell Polarization

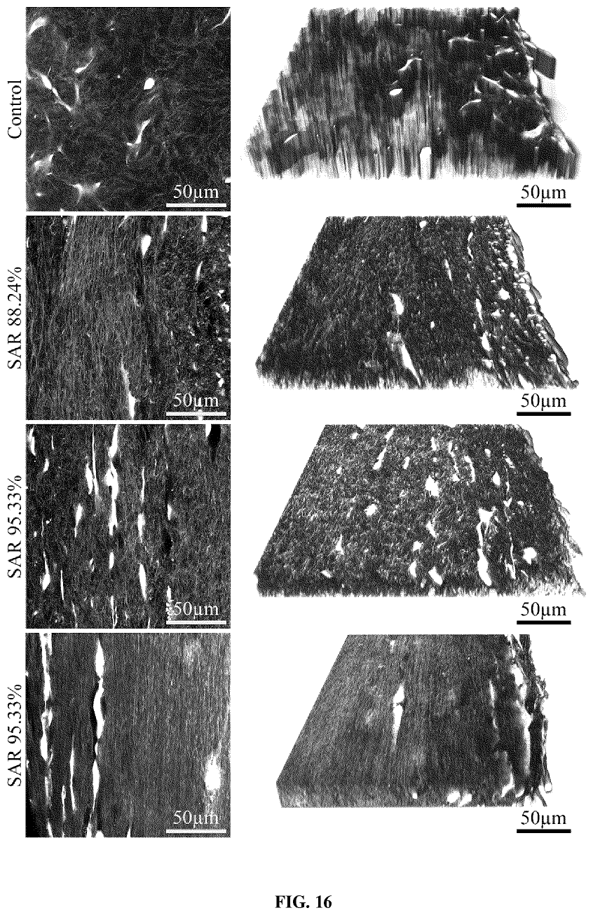

Cell polarization of the compacted biomaterials at day 3 in culture was investigated by staining paraffin-fixed thin sections of the cell-seeded biomaterials with HCS CellMask stain. Multiphoton confocal fluorescence microscopy was used to image the stained biomaterial sections based on second-harmonic generation. It was possible to image collagen fibrillar alignment and the stained cells at the same time (FIG. 16: 2D images on left hand side, and 3D reconstructed images on the right hand side). A loose collagen fibrillar architecture was observed where cells were randomly oriented in the biomaterial. With increasing compaction factor, the fibrillar structures appeared denser and cells appeared more polarized and stretched along the axis of aspiration. Cell polarization was also observed. This analysis revealed qualitatively how compaction factor value dictated the density of the solid phase of the biomaterials (CFD), the extent of fibrillar anisotropy as shown by SEM images and cell morphology as shown by the confocal microscopy images.

### Example 6: Compaction Factor is Related to Target Properties—Cell Distribution

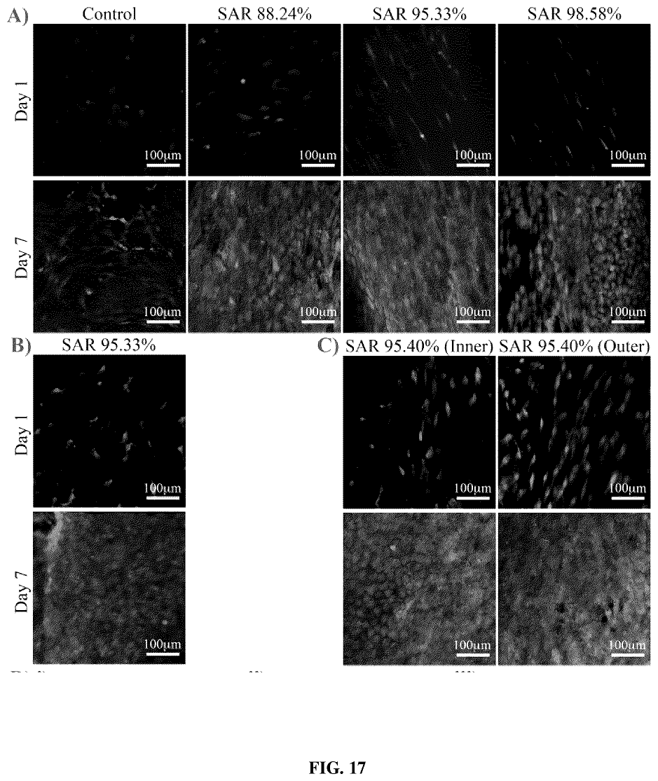

A uniform cell distribution throughout the volume of the compacted biomaterial is required for homogeneous tissue regeneration. In this regard, the compacted dense collagen gels demonstrated controlled cell seeding when examined up to 7 days in culture (using the calcein-AM staining method of Example 4). CLSM images demonstrated extensive cell viability and uniform distribution in all compacted biomaterials of different geometries (FIGS. 17A, 17B and 17C). Cell density appeared to be qualitatively higher in all compacted biomaterials at days 1 and 7 as a result of their compaction compared to that of the precursor biomaterial (hydrated collagen gel). Furthermore, viable cell distribution appeared to be more homogeneously distributed in the compacted biomaterials with compactor factor values between 88.24 and 95.33%. In addition, extensive cell viability near both the luminal and external surfaces of the tubular compacted biomaterials (FIG. 17C) confirmed the penetration of culture medium within the hollow structure. At day 1 in culture, and in contrast to the luminal surface, pockets of aligned cells were observed on the external surface of the tubular compacted biomaterials, which were influenced by the highly aligned architecture of the fibrils. SEM images captured from various surfaces of this cell-seeded tubular compacted biomaterials at day 7, confirmed the homogeneous spread of cells.

### Example 7: Compaction Factor is Related to Target Properties—Cell Viability & Metabolic Activity

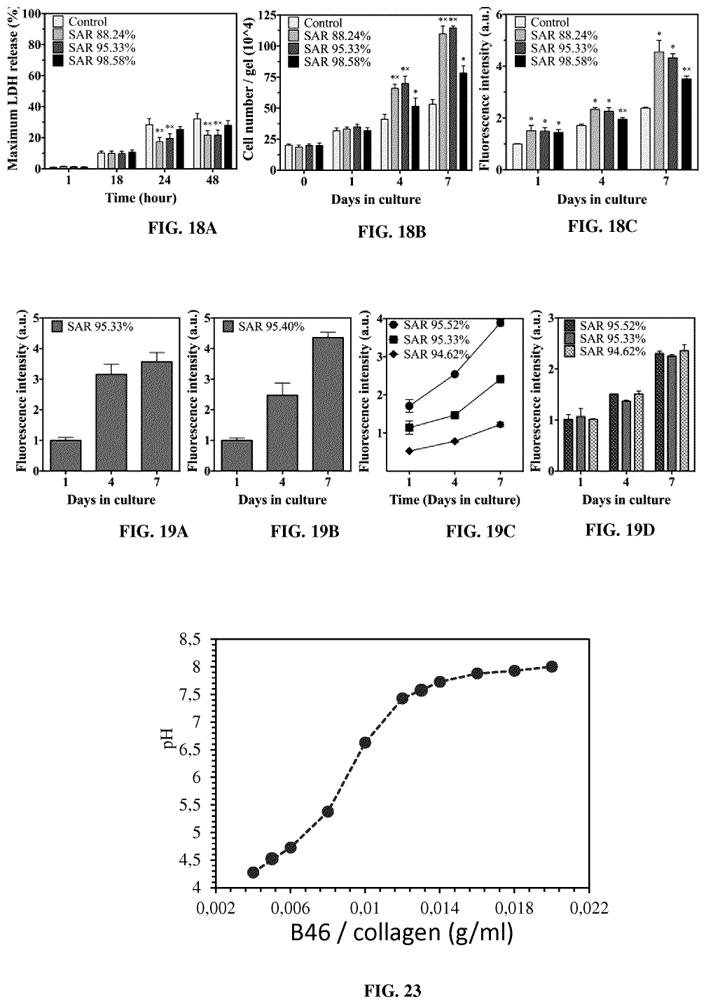

In order to investigate the effect of application of the compaction factor on seeded cell viability, cellular LDH release was measured from the compacted biomaterials up to 48 hours in culture and compared to that of the precursor biomaterial collagen gels (FIG. 18). Cell-free culture supernatant was collected from samples up to 48 h after the formation of the biomaterial and then incubated with the reaction mixture. LDH activity was determined by measuring the absorbance of the samples at 492 nm using a microplate reader and then compared to the total LDH release by 2.0×10⁵ cells killed in DMEM containing 1% Triton X-100. Results are expressed relative to maximum LDH release.

LDH release increased up to 24 hours in all compacted biomaterials but did not show any further increase at 48 hours. LDH release was significantly lower from cells seeded in compacted biomaterials of 88.24 and 95.33% compaction factor compared to those seeded in the precursor biomaterial control and the 98.58% compactor factor (^{x} p<0.05) (FIG. 18A). These results were also verified through quantification of cell viability and mortality. On the other hand, in compacted biomaterials of 98.58% compaction factor, cells were subjected to compaction into the biomaterial vessel post integration within the collagen matrix, and in this high solid phase weight % environment, cell-cell contact inhibition may significantly increase thus resulting in lower cell number (FIG. 18B). In contrast, in compact biomaterials with lower compaction factor values ranging between 88.24 and 95.33%, the compaction and shear forces applied were probably compatible with the slight displacement that cells can accommodate without affecting their viability and proliferation.

Seeded fibroblast metabolic activity measured up to 7 days in culture supported the CLSM images and indicated an increasing trend in all compacted biomaterials (FIG. 18C; FIGS. 19A, 19B, 19C and 19D). The metabolic activity of seeded fibroblasts as an indicator of cell viability and proliferation was evaluated using an alamarBlue® assay. The seeded fibroblasts were stained in growth medium with 10% alamarBlue® reagent and incubated under darkness in 5% CO₂ and 37° C. A fluorescent detection system was employed using a microplate reader. Background fluorescence measured in medium incubated with acellular gels was subtracted from all values. Data were normalized against the fluorescent intensity at day 1.

The compaction of the biomaterials resulted in a significantly (* p<0.05) higher metabolic activity compared to that of the precursor biomaterial. However, the metabolic activity of cells seeded in the compacted biomaterial with the highest compaction factor value (98.58%) was significantly lower (^{x} p<0.05) than those of the compacted biomaterials with compaction factor values ranging between 88.24 and 95.33% (FIG. 18C; FIGS. 19A, 19B, 19C and 19D). However, it was shown that increasing the CFD of compacted biomaterials up to 95.33% (FIG. 12), improved the cell spreading and proliferation. Remarkably, scaling-up or -down the dimensions of compacted biomaterials while maintaining both compaction factor and seeded cell density, resulted in different nominal values of metabolic activity, reflecting the different absolute number of cells seeded in the various compacted biomaterials (FIG. 19C). However, a similar metabolic activity trend was observed when values were normalized against the initial cell number (FIG. 19D). This confirmed that the rate of cell proliferation in compacted biomaterials can be tailored by controlling the compaction factor.

### Example 8: Compaction Factor is Related to Target Properties—Cell Remodelling

The extent of cell remodelling activity on the structure and mechanical properties of compacted biomaterials processed using compaction factors of 88.24 and 95.33% were compared up to 10 days in culture. Cell remodelling activity was investigated through determination of unconstrained, free-floating contraction of the formed biomaterials. Post processing, fibroblast seeded and acellular (control) 88.24% and 95.33% compacted biomaterials were cultured in 6 well culture plates and imaged at days 0 and 10.

Quantitative polymerase chain reaction (q-PCR) was conducted to amplify matrix metalloproteinases 1 a (Mmp1a), matrix metalloproteinases 13 (Mmp13), Tissue inhibitors of metalloproteinases 1 (Timp1) and collagen type I alpha 1 chain (Col1a1) transcripts as indicators of cell-induced remodelling activity using an RNA kit. This generated RNA transcripts that were reverse transcribed to cDNA by gScript™ cDNA synthesis kit (Quanta Bioscience Inc.) as per manufacturer instruction. PerfeCTa® SYBR® Green FastMix®^{ROX} (Quanta Bioscience Inc.) q-PCR master mix and primer pairs: Mmp1a forward: 5′-GTC TTT GAG GAG GAA GGC GAT ATT-3′, reverse: 5′-AGT TAG GTC CAT CAA ATG GGT TGT T-3′; Mmp13 forward: 5′-GGG CTC TGA ATG GTT ATG ACA TTC-3′, reverse: 5′-AGC GCT CAG TCT CTT CAC CTC TT-3′; Timp1 forward: 5′-GAC CTG ATC CGT CCA CAA AC-3′, reverse: 5′-GTG GGA AAT GCC GCA GAT ATC-3′; Gapdh forward: 5′-AAG GGC TCA TGA CCA CAG TC-3′, reverse: 5′-CAG GGA TGA TGT TCT GGG CA-3′ (300 nM each) were prepared for entry into the 7900HT q-PCR thermocycler (Applied Biosystems, USA). Cycling conditions were as follows: an initial denaturation of 95° C. for 10 min, followed by 40 repeats of 95° C. of denaturation for 15 s and an annealing/extension phase of 45 s. Using the 2^{-ΔΔCt} method, data was normalized to the expression of Gapdh and calibrated to the day 1 time point.

Tensile testing was carried out on fibroblast and acellular 88.24% compaction factor and 95.33% compaction factor formed biomaterials. Tensile testing at a displacement rate of 0.1 mm/s was carried out on specimens (n=5) at days 0 and 10 in culture using a Univert (mechanical testing frame) (CellScale Biomaterials, Canada) equipped with a 10 N load cell. The initial load-displacement data was processed to generate corresponding stress-strain curve by using the nominal biomaterial diameter (3.43 and 2.16 mm) and initial specimen length, respectively. The ultimate tensile strength (UTS) and the apparent modulus corresponded to the maximum load and slope of the linear region of the generated stress-strain curve.

The extent of contraction was lower in compacted biomaterials of 95.33% compaction factor compared to those of 88.24% compaction factor (FIGS. 20A and 20B). Accordingly, the expression of genes involved in cell-based remodelling exhibited distinct profiles when cells were seeded in compacted biomaterials of either 88.24 or 95.33% compaction factor (FIG. 20C). Expression of Mmp1 and Mmp13 was upregulated in fibroblasts seeded in 88.24% compaction factor at day 10 in culture suggesting that the cells were actively remodelling the compacted biomaterial. In contrast, the expression of tissue inhibitor metalloproteinase (Timp1) was upregulated at days 5 and 10 in fibroblasts when seeded in compacted biomaterials of 95.33% compaction factor. Furthermore, the expression of Col1a1 at day 5 was higher in cells when seeded in compacted biomaterial with 88.24% compaction factor than those in 95.33% compaction factor.

The effect of cell-based remodelling on the mechanical properties of the compacted biomaterials was investigated through tensile testing (FIGS. 20D and 20E). The stress-strain curves of acellular and cell-seeded compacted biomaterials were composed of three regions; an initial toe, a linear and a failure region. While the mechanical properties of the acellular and cell-seeded compacted biomaterials were similar at day 0, after 10 days in culture, cell-induced contraction in compacted biomaterials of 88.24% compaction factor resulted in a significant (p<0.05) increase in the solid phase weight %, which increased both the ultimate tensile strength (UTS) and apparent modulus (FIGS. 20D and 20E). In contrast, there were no significant differences observed in the mechanical properties and the solid phase weight % values of fibroblast seeded and acellular compacted biomaterials of 95.33% compaction factor at day 10 in culture (FIGS. 20D and 20E).

The ability to tune compacted biomaterials properties via varying the compaction factor led to significant modulation of extent of seeded cell remodelling activity. The structural changes observed in the compacted biomaterials of lower compaction factor (88.24%) value showed that the cell-generated forces induced a controlled, yet irreversible deformation in this compacted biomaterial, increasing its mechanical properties in line with the solid phase weight %. Here, it was demonstrated that compaction factor according to certain embodiments of the present technology can be used to effectively predict and tailor the temporal cellular responses within compacted biomaterials. Thus, through this approach, a pre-defined microenvironment can be designed and tuned along with controlling cellular remodelling activities to meet specific structural requirements of tissues, not only physiologically, but also and pathologically, thereby enabling high-throughput testing in drug discovery and safety screening. More widely, it may also impact the understanding of cancer diagnosis and treatment mechanisms, provide an animal-free platform in the safety and toxicology testing of chemicals and cosmetics, as well as advance stem cell research towards clinical applications in regenerative medicine.

### Example 9: Generating Dense Tubular Collagen Structures with a Continuous Body

Certain embodiments of the present technology were performed manually using biomaterial vessels with annular lumen to produce tubular shaped collagen biomaterials. The precursor biomaterial (highly hydrated collagen gel) was aspirated into the biomaterial vessel using a syringe pump and the formed collagen biomaterial was ejected from the biomaterial vessel by reversing the direction of the pressure induced by the syringe pump pressure.

TABLE 3
Dimensions of the double-walled capillary tubes and the resultant collagen tubes before and after drying.

| | | | Actual size of collagen tubes before | | Actual size of the collagen tubes | |
|---|---|---|---|---|---|---|
| | | | drying (a) | | after drying (b) | |
| | Outer diameter | Inner diameter | | internal | | internal |
| | (mm) | (mm) | thickness (mm) | diameter (mm) | thickness (mm) | diameter (mm) |
| 1 | 1.60 | 0.82 | 0.51 ± 0.05 | 0.72 ± 0.04 | 0.21 ± 0.03 | 0.66 ± 0.06 |
| 2 | 2.69 | 0.82 | 0.98 ± 0.04 | 0.64 ± 0.03 | 0.51 ± 0.05 | 0.64 ± 0.09 |
| 3 | 2.69 | 1.47 | 0.68 ± 0.05 | 1.19 ± 0.04 | 0.32 ± 0.05 | 1.21 ± 0.06 |

(a) Generated from optical microscope images of corresponding tubes by averaging 10 measurements.
(b) Generated from SEM images of corresponding tubes by averaging 10 measurements.
^{(c)} Calculated based on the thickness (t) of the tubes before and after drying.

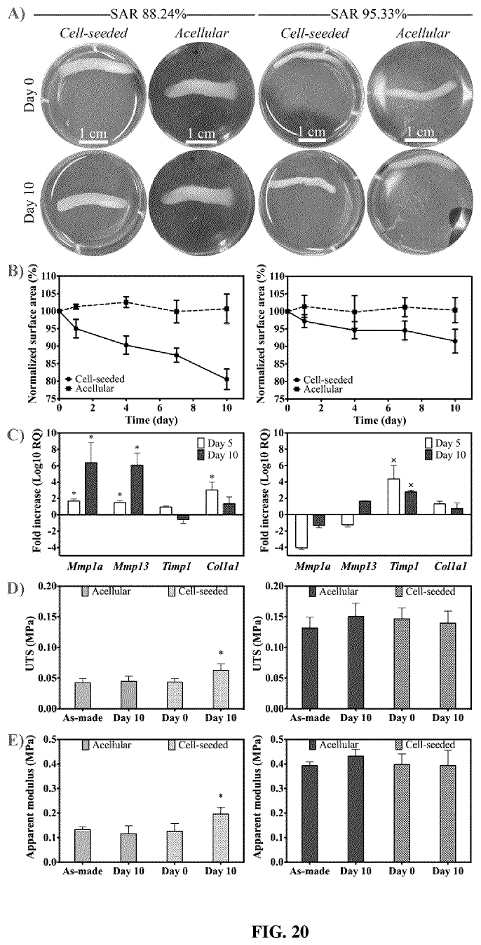

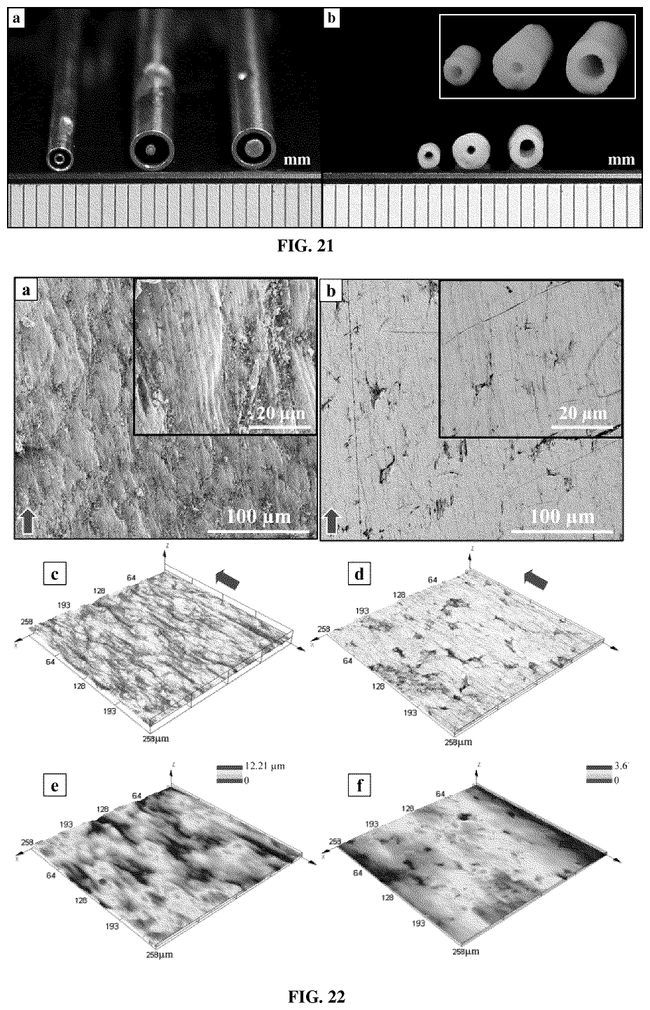

Scanning electron microscopy images of the resultant tubular biomaterials are shown in FIG. 21. The tubular biomaterials were of a continuous construction (no seams, no holes). The tubes had sufficient strength and integrity to maintain their shape even after ˜48-60% shrinkage during the drying process (Table 3).

### Example 10: Surface Roughness to Control Solid Phase Alignment

It was found that varying the surface roughness of an interior wall of the biomaterial vessel affected the solid phase alignment in the biomaterial which had been compacted in that biomaterial vessel. FIG. 22 shows SEM (a and b) and 3D confocal (c-f) images of example interior wall surfaces of the biomaterial vessels. The arrows indicate the direction of aspiration within the lumen of the needle.

Without being limited to the theory, a texture of the interior wall of the biomaterial vessel provides a continuous network of grip points preventing fibrillar slippage during the aspiration process, which consequently increase fibrillar alignment. In contrast, an interior wall with a lower surface roughness results in lower levels of fibrillar alignment compared to that of a rougher interior wall surface under the same compaction factor.

Thus, with sufficient surface roughness, solid phase alignment is directly related to the compaction factor value. However, if the interior surface has insufficient or no roughness, no fibril alignment can be obtained regardless of the compaction factor value of the system.

The relationship between surface roughness and alignment is expected to apply to a broader range of surface roughness values than those illustrated here.

Such tunable extents of fibrillar alignments in compacted biomaterials may be useful in the engineering of tendon, ligament, muscle and bone-like tissues where collagen fibril alignment is critical. Furthermore, by tailoring the surface roughness of the aspirating needles, an approach to successfully generate spatially tuned gradients in fibrillar alignments within T-DC compacted biomaterials may be achieved to mimic native conduit tissue/organs, e.g., by resulting in helicoidal fibrillar microstructures that exist within the walls of these tissues such as the aorta, an important requirement that is overlooked in other techniques of producing collagen-based tubular tissue structures.

### Example 11: Adjusting pH of Collagen Solution Affects its Viscoelastic Properties

The modulus of precursor biomaterial (hydrated collagen gel) were investigated using an apparatus for measuring viscoelastic properties of soft samples (ElastoSens™Bio², Rheolution Inc, US2016/0274015, the contents of which are incorporated herein. Briefly, the method comprises measuring the modulus as a function of time, during fibrillogenesis of the collagen solution). It was found that adjusting the pH of the starting collagen solution affected the modulus of the precursor biomaterial (pH range of 4 to 12 was investigated). Lowering the pH lowered the modulus, facilitating aspiration during the compaction step.

### Example 12: Making Mineralizable Collagen Biomaterials

A sol-gel derived borate glass derived formulation (46.1% B₂O₃-26.9% CaO-24.4% Na₂O-2.6% P₂O₅ in mol %; referred to as B46 in the figures) was incorporated into and dissolved in a precursor biomaterial, in this case a collagen solution. The borate glass formulation was made as previously reported (Lepry et al, Highly Bioactive Sol-Gel-Derived Borate Glasses, Chem. Mater. 27(13) (2015) 4821-4831; U.S. Ser. No. 15/317,746, the contents of which are hereby incorporated by reference).

More specifically, the borate particles were incorporated into collagen solutions as described here: Briefly, rat-tail tendon type I collagen solution (2.05 mg/mL, in 0.6% acetic acid) was added to suspensions of the borate glass particles in 10×-concentrated Dulbecco's Modified Eagle Medium until the borate glass particles dissolved. Different borate glass/collagen rations (e.g. 0.004, 0.006, . . . , and 0.02 g/mL) were prepared, and their pH measured at the end of the mixing process (n=3). The borate glass/collagen systems were gelled by placing in an incubator at 37° C. for ˜30 min. Note, that the usual step of collagen neutralization using NaOH was omitted.

The resultant highly hydrated collagen gels including the borate glass particles were then compacted using the compaction factors of the present technology (10G needle and aspiration of 0.15-0.25 μl/s). The compacted biomaterials were ejected at the rate of 2 μl/s into phosphate-buffered saline (PBS).

The borate glass formulation had the following properties:

| D50 | SSA | Pore Volume | Pore Width | Density |
|---|---|---|---|---|
| (μm) | (m²/g) | (cm³/g) | (nm) | (g/cm³) |
| 25.1 | 95.3 | 0.76 | 24.8 | 2.44 |

It was demonstrated that addition of the borate glass into the collagen solution affects the pH of the collagen solution (FIG. 23). It was also demonstrated that an adjustment of the pH towards physiological pH (7-8.5) induced fibrillogenesis (without requiring NaOH). Therefore, addition of controlled amounts of borate glass can be used to prepare the precursor biomaterial.

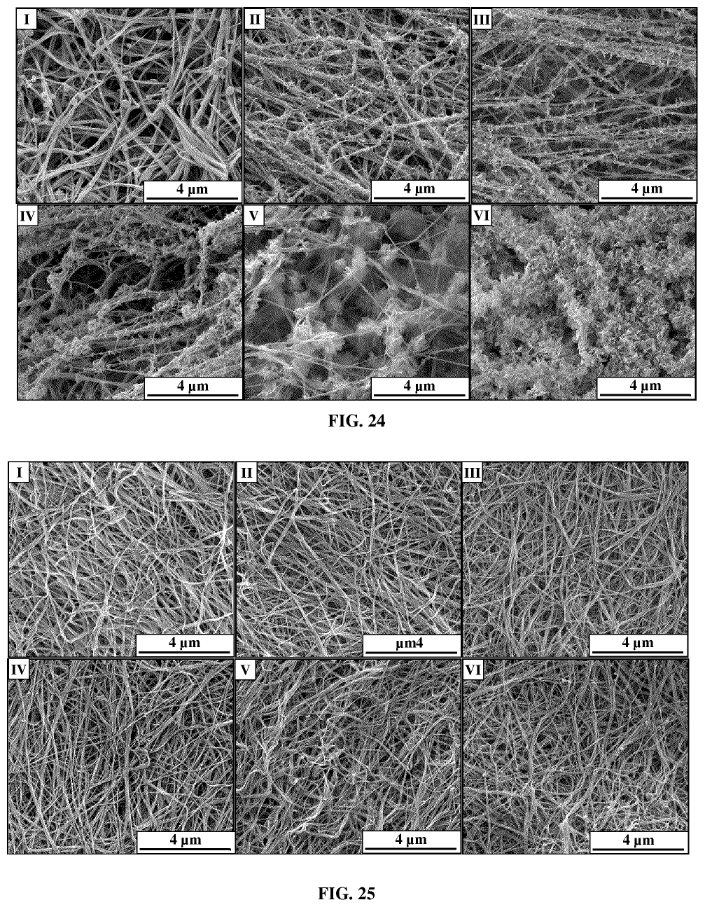

It was also demonstrated that the addition of the borate glass to the collagen solution induced mineralization in the dense collagen formed from the collagen solution. FIGS. 24 and 25 show borate glass at 0.013 g/mL and a collagen control (no borate) at various stages of SBF immersion: I) SBF (0 d), II) SBF (0.08 d), III) SBF (1 d), IV) SBF (3 d), V) SBF (7 d), and VI) SBF (14 d)). A homogenous network of nucleation sites were created on the collagen fibrils eventually leading to biomineralization to carbonated hydroxyapatite in simulated body fluid (SBF) within 2 hours. Mineralization was confirmed through Attenuated Total Reflectance Fourier Transform Infrared spectroscopy (ATR-FTIR), X-ray diffraction (XRD) and scanning electron microscopy (SEM).

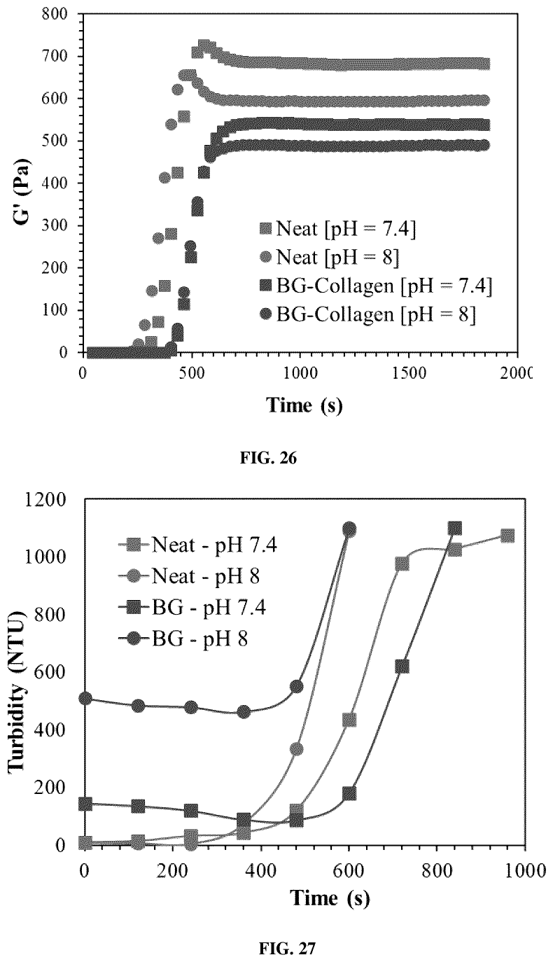

No change in the FTIR spectra of the control collagen gels was observed over 14 days immersion in SBF. However, for the functionalized gels (borate glass collagen gels), phosphate peak formation was observed as early as 2 hours immersion in SBF continuing up to 14 days, which is an indicator of hydroxyapatite (HA) formation/precipitation. The intensity of the phosphate peak is higher for the gels which contained more initial borate glass (pH 8) which may be the sign of more HA precipitation. It was observed by SEM that mineralization within the scaffolds was homogeneous—along the fibrils throughout the scaffold. It is believed that the released ions from the dissolution of borate glass particles induce fibrillization and simultaneously create a homogeneous network of nucleation sites on the fibrils. This is not believed to be the case when using other types of bioactive glasses such as silica-based ones. Fibrillogenesis/gelation of the control and hybridized biomaterials was monitored in two different manners: (1) monitoring shear storage modulus (G′) as a function of time using ElastoSens Bio2 (Rheolution) (FIG. 26), and (2) by observing change in turbidity of the systems over time using a turbidimeter (TB300 IR Turbidimeter (Orbeco Hellige)) (FIG. 27). The measurements were taken every 2 minutes at 37° C. until reaching a plateau or the limitation of the device. Note that the limit of our turbidimeter was 1100 Nephelometric Turbidity Units.

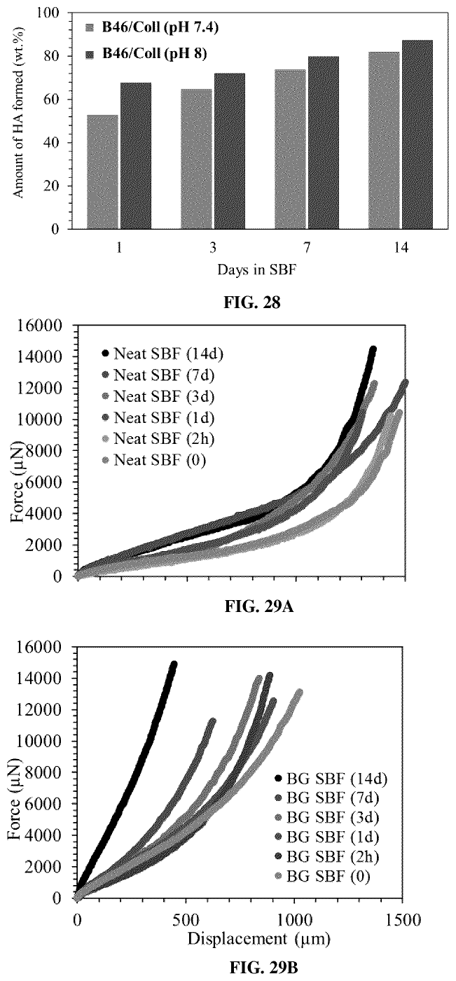

The change in weight of gels (after drying) over the incubation time in SBF was measured: There is no weight change for the control collagen gels up to 14 days immersion in SBF. However for the functionalized gels, the weight initially dropped which may be attributed to the dissolution of the remaining borate glass particles and then increased which is due to the formation/precipitation of CHA. Based on these results, the weight percentage of HA that is formed in the functionalized gels was calculated (FIG. 28). As can be seen, these scaffolds are heavily mineralized up to ˜80 wt % after 7-14 d immersion in SBF.

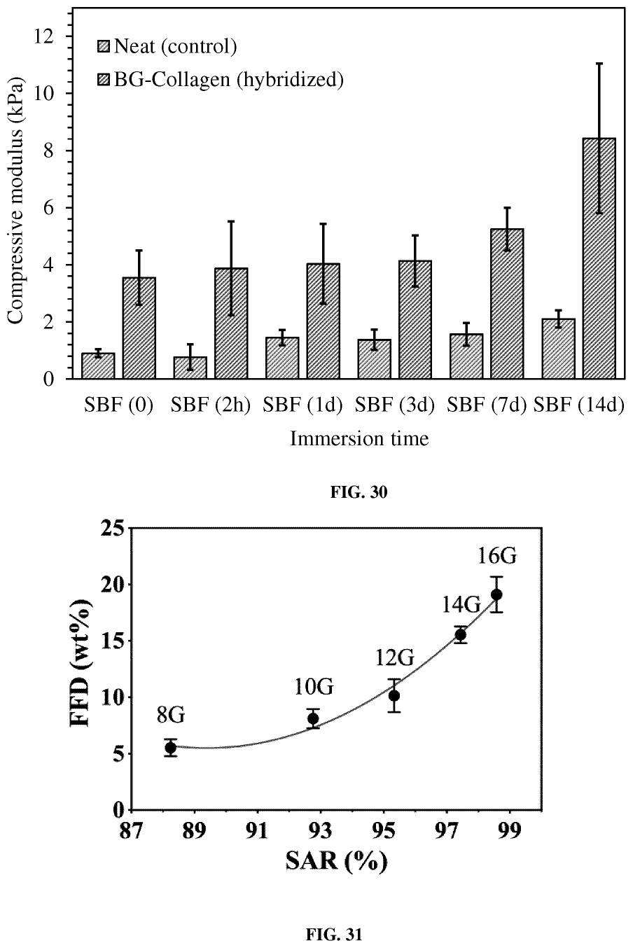

The effect on the mechanical properties of the gel was investigated. The gels were compressed using a microsquisher with a crosshead speed of 0.01 mm/s (Force vs. Displacement). The gels were cut into smaller pieces and placed between the two plates such that the “aligned fibrils” (i.e., the aspiration direction) are perpendicular to the plates (D=2.69 mm; which is the internal diameter of needle 10G). A small change was observed in the neat gels (control collagen gels) (FIG. 29A) after 14 days in SBF (the modulus slightly increased over time), whereas the modulus of the functionalized gels were higher and increased with longer incubation in SBF due to mineralization (FIG. 29B). This demonstrates the control of mechanical properties of a biomaterial through extent of mineralization/borate glass content. The slopes of the linear portion of the stress-strain curves were calculated (n=7) and are presented in FIG. 30 as the compressive modulus showing an improvement in compressive modulus of the hybridized samples due to the progressive mineralization in SBF over time, in particular after 14 days of immersion, whereas a minor increase can be observed for those of the non-hybridized samples.

This is a novel one-step process for a fast production of functionalized, compacted collagen scaffolds that are able to rapidly mineralize. The uses include bone tissue repair, augmentation or replacement.

Similarly, other bioactive agents can be added to the precursor biomaterial for mineralization such as anionic silk-fibroin derived peptides, non-collagenous proteins, anionic amino acids, calcium phosphate biomaterials. It is thought that in a cell seeded biomaterial with mineralizable properties, compaction factor will affect a rate of osteoblastic differentiation of seeded cells.

### Example 13: Compaction Factor Using Other Hydrogels

Embodiments of the present technology were applied to hydrogels other than collagen. More specifically, fibrin was used as the hydrogel to make compacted fibrin biomaterial using various compaction factors. Hybrid hydrogels of fibrin and collagen were also investigated. Collagen and hyaluronic acid were also investigated. Similar trends and results were observed for these hydrogels. For example, FIG. 31 shows an increase in fibrin fibrillar density (FFD) weight % with increasing compaction factor (SAR %) for a collagen-fibrin hybrid hydrogel. Therefore it can be appreciated that embodiments of the present technology are applicable to hydrogels other than collagen and which have a solid phase and a liquid phase. The solid phase can be fibrillar.

### Example 14: Different Target Properties in Tubular Biomaterials Made Using Different Compaction Factors—Vascular Smooth Muscle Cells

Cell seeded tubular collagen biomaterials with different target properties were made according to embodiments of the present technology using compaction factors of 83.99% and 94.58%. Precursor biomaterials (collagen solution) were seeded with vascular smooth muscle cells (VSMCs) after collagen solution neutralization at a density of 2×10⁵ cells/mL in different volumes, by transferring 0.35 and 1.5 of neutralized collagen solution in 96 and 48 well plates, respectively. Gelling was enabled in an incubator with 5% CO₂ atmosphere at 37° C. As cast, cell-seeded collagen gels (precursor biomaterial) were processed into the tubular structures as described in Example 9. The cell seeded tubular collagen biomaterials were monitored for up to 7 days in culture.

Confocal laser scanning microscopy (using Calcein-AM and EthD-1 staining) showed that the cells appeared viable in the cell seeded tubular collagen biomaterials at days 1 and 7. At day 7, the cell morphology in the 83.99% and 94.58% compaction factor made tubular biomaterials appeared different.

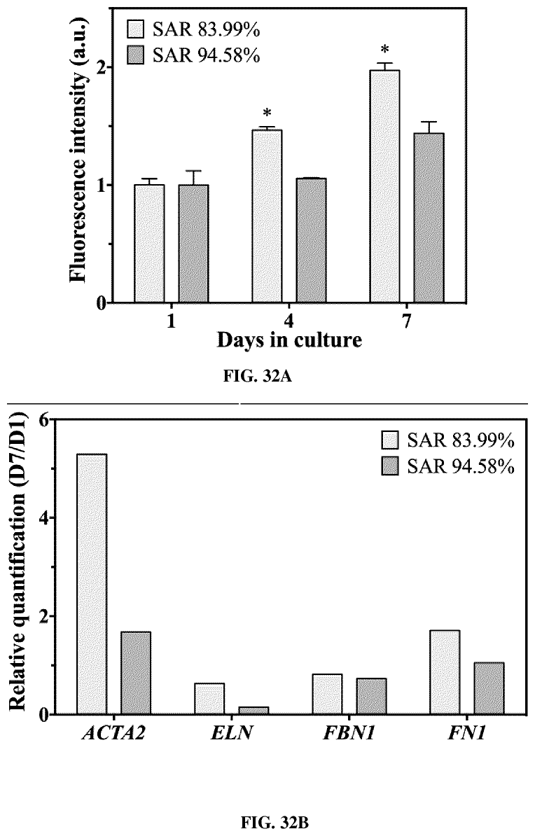

At days 4 and 7, the metabolic activity of the cells in the 83.99% compaction factor tubular biomaterial was significantly (p<0.05) higher than those in the 94.58% compaction factor tubular biomaterials (FIG. 32A). Gene expression of contractile markers in the cells of the different tubular biomaterials was assessed using using qRT-PCR system. The expression of Acta2, Eln, Fbn1, and Fn1 was higher in the cells of the 83.99% compaction factor made biomaterials compared to those of the 94.58% compaction factor made biomaterials (FIG. 32B).

### Example 15: Different Target Properties in Tubular Biomaterials Made Using Different Compaction Factors—Compressive Modulus of Acellular Vs Cellular Biomaterials

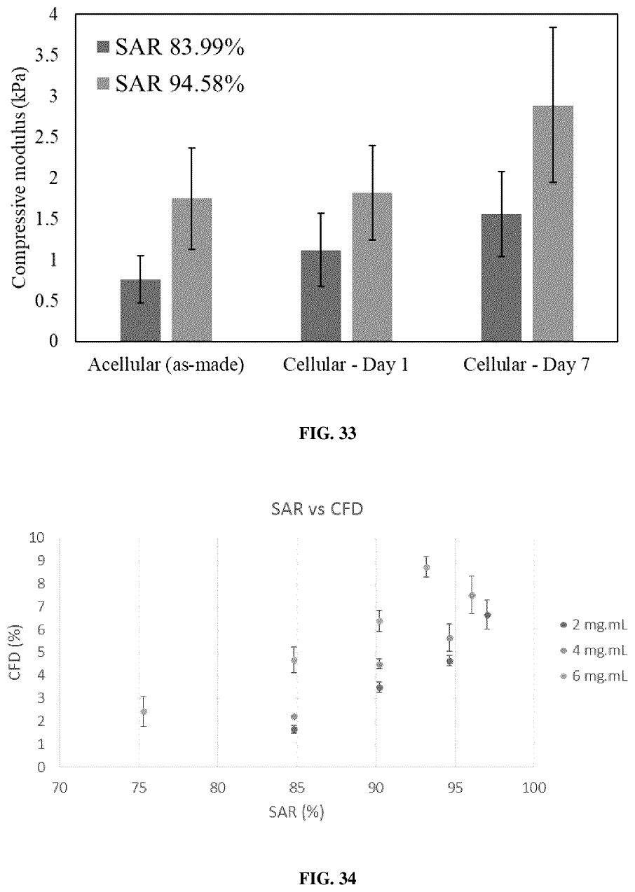

Cell seeded (vascular smooth muscle cells) and non-cell seeded collagen tubular biomaterials with different target properties were made according to embodiments of the present technology using compaction factors of 83.99% and 94.58%, and as described in Example 14. The compressive modulus of the samples were tested using a microsquisher (CellScale; Biomaterials Testing) with the displacement rate of 10 μm/s. These samples were kept hydrated prior to the subsequent static compression tests using the microsquisher. Samples were placed in the microsquisher such that their aspiration direction was parallel to the direction of the applied force. The compression tests were performed until a plateau was reached. Stress-strain curves were produced using the “force versus displacement” data given by the instrument and the initial cross-sectional areas and heights of the samples. Slopes of the linear portion of these stress-strain curves were calculated and presented as the compressive modulus of the samples. Table 4 and FIG. 33 demonstrate a relationship between one or more of the compaction factor, compressive modulus and incorporation of cells, and more specifically that increasing the compaction factor is related to an increase in compressive modulus in both cellular and acellular biomaterials.

TABLE 4
Compressive modulus in cellular and acellular tubular
biomaterials made using different compaction factors.

| | Compressive modulus in | Compressive modulus in Cellular | |
|---|---|---|---|
| Compaction | Acellular tubular | tubular biomaterials | |
| Factor (%) | biomaterials (as-made) | Day 1 | Day 7 |
| 83.99 | 0.76 ± 0.29 kPa | 1.12 ± 0.45 kPa | 1.56 ± 0.52 kPa |
| 94.58 | 1.75 ± 0.62 kPa | 1.82 ± 0.58 kPa | 2.89 ± 0.95 kPa |

### Example 16: Compaction Factor Related to Collagen Fibrillar Density and Collagen Concentration

Example 2 was repeated for collagen gels of differing concentration. It was seen that the relationship between compaction factor and collagen fibrillar density also applied for all concentrations of collagen gel that were tested (FIG. 34).

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A method for making a biomaterial comprising a hydrogel having a solid phase and liquid phase with a target property, the method being executed by a processor of a computer system operatively connectable to a bio-printing system for forming the biomaterial from a precursor biomaterial through a compaction process, the method comprising:
determining a compaction factor to be applied to the precursor biomaterial for forming the biomaterial based on a target property of the biomaterial, the compaction factor comprising a reduction in a given dimension of the precursor biomaterial relative to the given dimension in the formed biomaterial, the determining the compaction factor being based on a change in the property of the biomaterial with a change in the given dimension; and
determining one or more of a value of the given dimension of the precursor biomaterial and a value of the given dimension of the formed biomaterial based on the determined compaction factor.

2. The method of claim 1, further comprising sending instructions to the bio-printing system for forming the biomaterial based on the determined one or more of:
the determined value of the given dimension of the precursor biomaterial, and
the determined value of the given dimension of the formed biomaterial.

3. The method of claim 2, wherein the instructions cause the bio-printing system to aspirate at least a portion of the solid phase of the precursor biomaterial from a precursor biomaterial vessel into a biomaterial vessel to form the biomaterial with the reduction in the given dimension, the biomaterial vessel having a smaller value of the given dimension than a value of the given dimension of the precursor biomaterial vessel.

4. The method of claim 3, further comprising sending instructions to the bio-printing system to eject the formed biomaterial from the biomaterial vessel.

5. The method of claim 4, further causing one or more of an x-direction, a y-direction or a z-direction of movement of the biomaterial vessel during its ejection.

6. The method of claim 1, further comprising causing one or both of: selection of a given precursor biomaterial vessel from a kit of precursor vessels, the given precursor biomaterial vessel having the determined value of the given dimension of the precursor biomaterial; and selection of a given biomaterial vessel from a kit of biomaterial vessels, the given biomaterial vessel having the determined value of the given dimension of the formed biomaterial.

7. (canceled)

8. The method of claim 1, further comprising receiving input of the target property of the biomaterial.

9. The method of claim 1, further comprising receiving input of a target value of the given dimension of the precursor biomaterial, and determining a value of the given dimension of the biomaterial based on the determined compaction factor.

10. The method of claim 1, wherein the target property is one or more of:
an extent of alignment of a solid phase in the biomaterial,
a content of the aligned phase in the biomaterial,
a content of the solid phase in the biomaterial,
a distribution of the aligned phase in the biomaterial,
a mechanical property of the biomaterial, and
a cell-independent contraction property of the biomaterial.

11. The method of claim 1, wherein the biomaterial has cells incorporated therein, and the target property is one or more of:
an orientation of the cells incorporated in the biomaterial,
an alignment of the cells incorporated in the biomaterial,
a distribution of the cells in the biomaterial,
cell activity in the biomaterial, and
a cell-induced contraction property of the biomaterial.

12. The method of claim 1, wherein the given dimension is one or more of:
a cross-sectional surface area of the precursor biomaterial and the biomaterial;
a diameter of the precursor biomaterial and the biomaterial;
a volume of the precursor biomaterial and the biomaterial;
a surface area of a precursor biomaterial vessel in contact with the precursor biomaterial; and
a surface area of a biomaterial vessel in contact with the biomaterial.

13. The method of claim 1, further comprising causing the display on a screen associated with the of one or more of: the determined compaction factor, the determined value of the given dimension of the precursor biomaterial, and the determined value of the given dimension of the formed biomaterial based.

14. The method of claim 1, wherein the compaction factor is less than about 98.6% reduction in a cross-sectional surface area of the precursor biomaterial compared to the cross-sectional surface area of the formed biomaterial, and optionally between about 88% and 98.6% reduction in the cross-sectional surface area of the precursor biomaterial compared to the cross-sectional surface area of the formed biomaterial.

15-20. (canceled)

21. The method of claim 1, wherein the method of forming the biomaterial comprises reducing the given dimension of the precursor biomaterial whilst allowing fluid expulsion from the precursor biomaterial to form the biomaterial.

22. The method of claim 1, wherein the given dimension is a cross-sectional area of the precursor biomaterial in a precursor biomaterial vessel, and reducing the given dimension comprises causing the precursor biomaterial to flow from the precursor biomaterial vessel into a biomaterial vessel, the biomaterial vessel having a smaller cross-sectional diameter than the precursor biomaterial vessel.

23. The method of claim 1, wherein the given dimension is a cross-sectional area, and the compaction factor comprises (a cross-sectional area value of the precursor biomaterial minus a cross-sectional area value of the formed biomaterial)/the cross-sectional area value of the precursor biomaterial×100.

24. The method of claim 1, wherein the biomaterial comprises one or more hydrogels selected from: collagen, hyaluronan, chitosan, fibrin, gelatin, silk fibroin, alginate, agarose, chondroitin sulphate, polyacrylamide, polyethylene glycol (PEG), poly vinyl alcohol (PVA), polyacrylic acid (PAA), hydroxy ethyl methacrylate (HEMA), polyanhydrides, poly(propylene fumarate) (PPF).

25. A system for making a biomaterial comprising a hydrogel having a solid phase and liquid phase with a target property, the system comprising:
a computer system having a processor and operatively connectable to a bio-printing system, the processor arranged to execute a method comprising:
determining a compaction factor to be applied to the precursor biomaterial for forming the biomaterial based on a target property of the biomaterial, the compaction factor comprising a reduction in a given dimension of the precursor biomaterial relative to the given dimension in the formed biomaterial, the determining the compaction factor being based on a change in the property of the biomaterial with a change in the given dimension; and
determining one or more of a value of the given dimension of the precursor biomaterial and a value of the given dimension of the formed biomaterial based on the determined compaction factor.

26. The system of claim 25, further comprising the bio-printing system and wherein the bio-printing system comprises:
a pump module for applying a pressure to a precursor biomaterial to compact the precursor biomaterial into a biomaterial vessel, and optionally a stage module for enabling relative movement between the precursor biomaterial and the biomaterial.

27. The system of claim 25, further comprising:
a precursor biomaterial vessel for holding a precursor biomaterial, and
a biomaterial vessel for compacting the precursor biomaterial therein.

28-59. (canceled)
